(19) United States
(12) Patent Application Publication
(10) Pub. No.: US 2015/0252377 A1
(43) Pub. Date: Sep. 10, 2015

(54) GENES CONTROLLING PHOTOPERIOD SENSITIVITY IN MAIZE AND SORGHUM AND USES THEREOF

(71) Applicants: Olga N. DANILEVSKAYA, (US); Xin MENG, (US); PIONEER HI-BRED INTERNATIONAL, INC., Johnston, IA (US); E. I. DUPONT DE NEMOURS & COMPANY, Wilmington, DE (US)

(72) Inventors: Nathan David Coles, Woodland, CA (US); Olga Nikolaevna Danilevskaya, Johnston, IA (US); Xin Meng, Johnston, IA (US)

(21) Appl. No.: 14/431,386

(22) PCT Filed: Oct. 1, 2013
(86) PCT No.: PCT/US2013/062806

§ 371 Date: Mar. 26, 2015

Related U.S. Application Data
(60) Provisional application No. 61/709,239, filed on Oct. 3, 2012.
(60) Provisional application No. 61/771,193, filed on Mar. 1, 2013.
(60) Provisional application No. 61/776,846, filed on Mar. 12, 2013.

(51) Int. Cl. C12N 15/82; C07K 14/415; C12Q 1/68

(52) U.S. Cl. CPC C12N 15/8222; C12N 15/827; C12N 15/8273; C12N 15/8279; C12N 15/8286; C12Q 1/6895; C07K 14/415; C12Q 2600/13; C12Q 2600/156

(57) ABSTRACT

The present disclosure provides polynucleotide sequences controlling the photoperiod sensitive trait in maize. The disclosure provides polynucleotide sequences and the use of encoded polypeptides associated with the photoperiod sensitivity. The disclosed sequences are responsible for controlling plant growth, source-sink relationships and yield in crop plants.

### TECHNICAL FIELD

The present disclosure relates generally to plant molecular biology. More specifically, it relates to nucleic acids and methods for modulating their expression thereby controlling photoperiod sensitivity, flowering time and flower/seed production in plants.

### BACKGROUND OF THE DISCLOSURE

Flowering time is an important agronomic trait because it determines the growing region of adaptation. Most plant species are induced to flower in response to environmental stimuli such as a day length and temperature. Corn was domesticated in Mexico from its wild ancestor, teosinte (Zea mays ssp. parviglumis) which requires short days for flowering. The modern agronomically important temperate corn is a day-neutral plant as the photoperiod sensitivity has been lost during artificial selection by humans for corn adaptation to the northern latitudes under the long summer days. The maize tropical lines still retain a short-day requirement for flowering.

Maize germplasms from tropical and sub-tropical lines are a natural source of important agronomic traits such as disease and insect resistance as well tolerance to abiotic stresses. The tropical germplasm is used for broadening the genetic diversity of the temperate corn in breeding programs for drought tolerance and yield improvement. However, photoperiod sensitivity of tropical and subtropical varieties hinders the free and rapid exchange of germplasm across latitudes and presents a problem in breeding programs in temperate areas. Photoperiodism has been the major constrain in evaluation of tropical materials for temperate areas. Due to photoperiodism tropical cultivars grown in the temperate environments are characterized by late flowering, a tall stature, larger leaves, larger tassels and lower grain yield (Hallauer and Carena, in Handbook of Plant Breeding, Cereals, Springer 2009). To overcome these problems mass selection is used in breeding for earliness in populations produced by crosses between tropical and temperate cultivars. However during the empirical selection for earliness the significant amount of the tropical germplasm could be lost and might eliminated valuable alleles. This would detract from the original goals of increasing genetic diversity and introducing useful alleles from exotic tropical materials. This problem may be overcome by using the marker assisted selection (MAS) or gene-assisted selection (GAS) if genes controlling photoperiod sensitivity are identified.

At the molecular level the current knowledge of the photoperiod sensitivity control in maize is very limited. This disclosure identifies a gene, ZmCCT10 (stands for Zea mays CCT domain on chromosome 10) which functions as a major regulator of the photoperiod response in tropical maize. In tropical lines ZmCCT10 is highly expressed in the diurnal manner under non-inductive long days, but its expression is not detected under inductive short days. Hence ZmCCT10 functions through repressing flowering time genes under long days but not short days in the tropical maize.

In temperate cultivars ZmCCT10 has lost control over the flowering time because of its low expression which resulted in insensitivity to the day length. This disclosure identifies genetic variations in the promoter sequence of the tropical ZmCCT10 alleles that are associated with photoperiod sensitivity. These genetic variations can be used for marker assisted selection or gene-assisted selection in breeding programs exploiting the tropical or exotic germplasms.

This disclosure proposes the down-regulation of ZmCCT10 in topical hybrids via various transgenic cassettes to override the short-day requirement of tropical germplasms that will allow screening for agronomical valuable traits using directly the first generation F1 hybrids.

### BRIEF DESCRIPTION OF THE DRAWINGS

FIG. 1. The ZmCCT10 gene model is composed of two exons and the 2.3-kb promoter region. There are two polymorphic regions in the promoter that are variable between tropical, temperate lines and wild maize progenitor teosinte.

FIG. 2 (2A-2B). Polypeptide alignments of five closely related CCT proteins from maize, teosinte, rice and sorghum. Three conservative domains are framed. The first and second domains are novel domains of unknown function. The 43 amino acid CCT domain is located at C-terminus implying the protein function in light signal transduction. The putative DNA/RNA binding motif C-X₂-C-Xₙ-H-X₂-H is marked by asterisks.

FIG. 3. Diurnal expression of ZmCCT10 and ZCN8 (the activator of flowering) in leaves of temperate and tropical lines. Under permissive short days ZmCCT10 transcription is very low in tropical lines, whereas ZCN8 expression is very high with the peak of expression at dawn. Under non-permissive long days ZmCCT10 expression is high with the peak of expression later in the morning and ZCN8 transcription is very low. The opposite rhythmic transcription suggests that ZmCCT10 functions as a repressor of ZCN8 in tropical lines. In the day neutral temperate maize, ZmCCT10 transcription is low under short and long days. Consistently ZCN8 expression is higher under both conditions in temperate lines further supporting ZmCCT10 function as a photoperiod regulator in maize.

FIG. 4. Transgenic plants over-expressing ZmCCT10 in the temperate Gaspe line show the typical tropical plant architecture: more leaves, longer and wider leaves, thicker stem and appearance of brace roots in internodes.

### BRIEF SUMMARY OF THE DISCLOSURE

The current research of maize photosensitivity is based on QTL (Quantitative Trait Locus) mapping for flowering time. Four major photoperiod sensitivity loci were identified and a major QTL was mapped to chromosome 10. This disclosure describes the isolation of cDNA of the QTL on chromosome 10 and designated as gene ZmCCT10. Phylogenic analysis of the putative protein revealed its close homology to the rice Ghd7, which is the repressor of flowering under the long days.

This disclosure provides evidence that ZmCCT10 does function as a repressor of flowering time in the tropical lines. In temperate maize ZmCCT10 is not expressed under both day-length that resulted in a day-neutral habit. Two homologue genes were identified in the sorghum genome. Sorghum is a short-day plant. These genes were named SbCCT1 and SbCCT6 which stands for Sorghum bicolor CCT chromosome 1 and chromosome 6. Over-expression of the ZmCCT10 gene in a temperate maize produced transgenic plants with a tropical architecture.

Transgenic manipulation of ZmCCT10 in maize or SbCCT1 and SbCCT6 in sorghum will allow to reduce photoperiod sensitivity and flowering times and in breeding programs. Specifically down-regulation of maize or sorghum CCT10 genes in the F1 hybrids can be employed to enhance the free and rapid exchange of germplasm across latitudes.

A method of screening a population of plants for improved tolerance to biotic or abiotic stress, the method includes:

a. providing a population of plants, wherein the photoperiod sensitivity of the plants is altered due to the down regulation of an endogenous gene, wherein the endogenous gene encodes a protein that comprises a CONSTANS, CONSTANS-LIKE, TOC1 (CCT) domain; and

b. screening the population of plants for the improved tolerance to biotic or abiotic stress.

In an embodiment, the population of plants with reduced CCT gene expression is grown in a plant growing environment that is different than a normal growing environment for the population of plants in the absence of the down regulation of the endogenous gene. In an embodiment, the endogenous gene encodes a polypeptide that comprises a CCT domain that is at least 90% identical to the CCT domain of SEQ ID NO: 3.

A method of creating a heterotic breeding population of maize plants, the method includes:

a. providing a transgenic parent and a photoperiod sensitive parent, wherein the transgenic parent comprises a recombinant genetic element that is capable of reducing the expression of a photoperiod sensitivity gene of the photoperiod sensitive parent, and

b. crossing the transgenic parent with the photoperiod sensitive parent, thereby producing a heterotic breeding population.

In an embodiment, the recombinant genetic element down regulates an endogenous gene that encodes a polypeptide that comprises a CCT domain that is at least 90% identical to the CCT domain of SEQ ID NO: 3.

In an embodiment, the transgenic parent is a transgenic female parent, and the photoperiod sensitive parent is a non-transgenic male parent.

In an embodiment, the transgenic parent is day-length neutral with respect to photoperiod sensitivity.

In an embodiment, the photoperiod sensitive parent is a tropical line.

In an embodiment, progeny is selected from crossing the photoperiod sensitive parent with the transgenic parent for enhanced tolerance to a biotic or an abiotic stress.

In an embodiment, the abiotic stress is drought.

In an embodiment, the biotic stress tolerance is insect resistance or disease resistance.

A method of screening for allelic variations of a gene encoding ZmCCT10 polypeptide comprising a CONSTANS, CONSTANS-LIKE, TOC1 (CCT) domain, the method includes sequencing the genomic region of the maize genome that regulates the expression level of ZmCCT10 polypeptide and identifying one or more allelic variations within the regulatory region of the ZmCCT10 gene that are correlated with reduced expression of ZmCCT10 mRNA.

A method of modulating photoperiod sensitivity in a plant, the method includes:

a. providing a photoperiod sensitive plant with a recombinant polynucleotide, the polynucleotide operably linked to a promoter, wherein the polynucleotide down regulates the expression of an endogenous gene encoding a polypeptide comprising a CONSTANS, CONSTANS-LIKE, TOC1 (CCT) domain;

b. reducing the photoperiod sensitivity of the plant by downregulating the expression endogenous gene.

In an embodiment, the plant is from a plant selected from the group consisting of: maize, soybean, sunflower, sorghum, canola, wheat, alfalfa, cotton, rice, barley, millet, peanut, sugar cane and cocoa.

### DETAILED DESCRIPTION OF THE DISCLOSURE

Unless defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

The present disclosures now will be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, BOTANY: PLANT BIOLOGY AND ITS RELATION TO HUMAN AFFAIRS, John Wiley (1982); CELL CULTURE AND SOMATIC CELL GENETICS OF PLANTS, vol. 1, Vasil, ed. (1984); Stanier, et al., THE MICROBIAL WORLD, 5^{th} ed., Prentice-Hall (1986); Dhringra and Sinclair, BASIC PLANT PATHOLOGY METHODS, CRC Press (1985); Maniatis, et al., MOLECULAR CLONING: A LABORATORY MANUAL (1982); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984) and the series METHODS IN ENZYMOLOGY, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), 0-Beta Replicase systems, transcription-based amplification system (TAS) and strand displacement amplification (SDA). See, e.g., DIAGNOSTIC MOLECULAR MICROBIOLOGY: PRINCIPLES AND APPLICATIONS, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, PROTEINS, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “CCT nucleic acid” means a nucleic acid comprising a polynucleotide (“CCT polynucleotide”) encoding a CCT polypeptide.

As used herein, “nucleic acid” includes reference to a deoxyribonucleotide or ribonucleotide polymer in either single- or double-stranded form and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, GUIDE TO MOLECULAR CLONING TECHNIQUES, from the series METHODS IN ENZYMOLOGY, vol. 152, Academic Press, Inc., San Diego, Calif. (1987); Sambrook, et al., MOLECULAR CLONING: A LABORATORY MANUAL, 2^{nd} ed., vols. 1-3 (1989) and CURRENT PROTOCOLS IN MOLECULAR BIOLOGY, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” includes reference to bushels per acre of a grain crop at harvest, as adjusted for grain moisture (15% typically). Grain moisture is measured in the grain at harvest. The adjusted test weight of grain is determined to be the weight in pounds per bushel, adjusted for grain moisture level at harvest. As used herein, improved “source-sink” relationship includes reference to a trait associated with an improvement of the ratio of assimilate supply (i.e., source) and demand (i.e., sink) during grain filling.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins (e.g., transcription factors) to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions.

As used herein, “regulatory element” or “regulatory polynucleotide” refers to nucleic acid fragment that modulates the expression of a transcribable polynucleotide that is associated with the regulatory element. Such association can occur in cis. A plant promoter can also be used as a regulatory element for modulating the expression of a particular gene or genes that are operably associated to the promoters. When operably associated to a transcribable polynucleotide molecule, a regulatory element affects the transcriptional pattern of the transcribable polynucleotide molecule. “cis-element” or “cis-acting element” refers to a cis-acting transcriptional regulatory element that affects gene expression. A cis-element may function to bind transcription factors, trans-acting proteins that modulate transcription. The CCT promoters disclosed herein may contain one or more cis-elements that provide CCT gene expression pattern.

The plant promoters and the regulatory elements disclosed herein can include nucleotide sequences generated by promoter engineering, i.e., combination of known promoters and/or regulatory elements to produce artificial, synthetic, chimeric or hybrid promoters. Such promoters can also combine cis-elements from one or more promoters, for example, by adding a heterologous tissue specific regulatory element to a promoter that contains CCT expression regulatory elements. Thus, the design, construction, and use of chimeric or hybrid promoters comprising at least one cis-element of the promoters disclosed herein for modulating the expression of operably linked polynucleotide sequences is contemplated.

The promoters are contemplated for use in modulating the expression pattern of one or more heterologous genes. The term “heterologous” in this context means that the expression of the nucleotide of interest is modulated by a promoter sequence or a fragment thereof that is not the nucleotide's own promoter. Deletion constructs of the various promoter sequences disclosed herein are readily made by one of ordinary skill in the art following the guidance provided herein. About 25-50 contiguous nucleotides that flank the 3′ or the 5′ ends of the disclosed regulatory elements are selected for modulation of gene expression. Mutational analyses are also performed to enhance the specificity of CCT regulation.

The term “CCT polypeptide” refers to one or more amino acid sequences. The term is also inclusive of fragments, variants, homologs, alleles or precursors (e.g., preproproteins or proproteins) thereof. A “CCT protein” comprises a CCT polypeptide. Unless otherwise stated, the term “CCT nucleic acid” means a nucleic acid comprising a polynucleotide (“CCT polynucleotide”) encoding a CCT polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem. 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part I, chapter 2, “Overview of principles of hybridization and the strategy of nucleic acid probe assays,” Elsevier, New York (1993) and CURRENT PROTOCOLS IN MOLECULAR BIOLOGY, chapter 2, Ausubel, et al., eds, Greene Publishing and Wiley-Interscience, New York (1995). Unless otherwise stated, in the present application high stringency is defined as hybridization in 4× SSC, 5×Denhardt's (5 g Ficoll, 5 g polyvinypyrrolidone, 5 g bovine serum albumin in 500 ml of water), 0.1 mg/ml boiled salmon sperm DNA and 25 mM Na phosphate at 65° C. and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package®, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The disclosure discloses CCT polynucleotides and polypeptides. The novel nucleotides and proteins of the disclosure have an expression pattern which indicates that they regulate photoperiod sensitivity and thus play an important role in plant development. The polynucleotides are expressed in various plant tissues. The polynucleotides and polypeptides thus provide an opportunity to manipulate plant development to alter seed and vegetative tissue development, timing or composition. This may be used to create a sterile plant, a seedless plant or a plant with altered

### Nucleic Acids

The present disclosure provides, inter alia, isolated nucleic acids of RNA, DNA and analogs and/or chimeras thereof, comprising a CCT polynucleotide.

The present disclosure also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants is listed in Table 4 of Murray, et al., supra.

The CCT nucleic acids of the present disclosure comprise isolated CCT polynucleotides which are inclusive of:
(a) a polynucleotide encoding a CCT polypeptide and conservatively modified and polymorphic variants thereof;
(b) a polynucleotide having at least 70% sequence identity with polynucleotides of (a) or (b);
(c) complementary sequences of polynucleotides of (a) or (b).
   The following table, Table 1, lists the specific identities of the polynucleotides and polypeptides and disclosed herein.

TABLE 1

| | | Polynucleotide/ | |
|---|---|---|---|
| Name | Plant species | Polypeptide | SEQ ID NO: |
| ZmCCT10-B73 | Zea mays | Polynucleotide | SEQ ID NO: 1 |
| | | genomic | |
| ZmCCT10-B73 | Zea mays | Polynucleotide | SEQ ID NO: 2 |
| | | cDNA | |
| ZmCCT10-B73 | Zea mays | Polypeptide | SEQ ID NO: 3 |
| ZmCCT10-Gaspe | Zea mays | Polynucleotide | SEQ ID NO: 4 |
| | | genomic | |
| ZmCCT10-Gaspe | Zea mays | Polynucleotide | SEQ ID NO: 5 |
| | | cDNA | |
| ZmCCT10-Gaspe | Zea mays | Polypeptide | SEQ ID NO: 6 |
| ZmCCT10-DCBNF | Zea mays | Polynucleotide | SEQ ID NO: 7 |
| | | genomic | |
| ZmCCT10-DCBNF | Zea mays | Polynucleotide | SEQ ID NO: 8 |
| | | cDNA | |
| ZmCCT10-DCBNF | Zea mays | Polypeptide | SEQ ID NO: 9 |
| ZmCCT10-BC26N | Zea mays | Polynucleotide | SEQ ID NO: 10 |
| | | genomic | |
| ZmCCT10-BC26N | Zea mays | Polynucleotide | SEQ ID NO: 11 |
| | | cDNA | |
| ZmCCT10-BC26N | Zea mays | Polypeptide | SEQ ID NO: 12 |
| ZmCCT10-CML436 | Zea mays | Polynucleotide | SEQ ID NO: 13 |
| | | genomic | |
| ZmCCT10-CML436 | Zea mays | Polynucleotide | SEQ ID NO: 14 |
| | | cDNA | |
| ZmCCT10-CML436 | Zea mays | Polypeptide | SEQ ID NO: 15 |
| SbCCT1 | Sorghum bicolor | Polynucleotide | SEQ ID NO: 16 |
| | | genomic | |
| SbCCT1 | Sorghum bicolor | Polynucleotide | SEQ ID NO: 17 |
| | | cDNA | |
| SbCCT1 | Sorghum bicolor | Polypeptide | SEQ ID NO: 18 |
| SbCCT6 | Sorghum bicolor | Polynucleotide | SEQ ID NO: 19 |
| | | genomic | |
| SbCCT6 | Sorghum bicolor | Polynucleotide | SEQ ID NO: 20 |
| | | cDNA | |
| SbCCT6 | Sorghum bicolor | Polypeptide | SEQ ID NO: 21 |
| Primer Forward | Zea mays | Polynucleotide | SEQ ID NO: 22 |
| Primer Reverse | Zea mays | Polynucleotide | SEQ ID NO: 23 |
| Primer Forward | Zea mays | Polynucleotide | SEQ ID NO: 24 |
| Primer Reverse | Zea mays | Polynucleotide | SEQ ID NO: 25 |
| Primer Forward | Zea mays | Polynucleotide | SEQ ID NO: 26 |
| Primer Reverse | Zea mays | Polynucleotide | SEQ ID NO: 27 |
| Primer Forward | Zea mays | Polynucleotide | SEQ ID NO: 28 |
| Primer Reverse | Zea mays | Polynucleotide | SEQ ID NO: 29 |
| Primer Forward | Zea mays | Polynucleotide | SEQ ID NO: 30 |
| Primer Reverse | Zea mays | Polynucleotide | SEQ ID NO: 31 |
| Probe | Zea mays | Polynucleotide | SEQ ID NO: 32 |
| Insert in transgenic | Zea mays | Polynucleotide | SEQ ID NO: 33 |
| vector | | | |
| ZmCCT10-parviglumis | Zea mays spp parviglumis | Polynucleotide | SEQ ID NO: 34 |
| | | genomic | |
| ZmCCT10-parviglumis | Zea mays spp parviglumis | Polynucleotide | SEQ ID NO: 35 |
| | | cDNA | |
| ZmCCT10-parviglumis | Zea mays spp parviglumis | Polypeptide | SEQ ID NO: 36 |
| ZmCCT10-luxurians | Zea luxurians | Polynucleotide | SEQ ID NO: 37 |
| | | genomic | |
| ZmCCT10-luxurians | Zea luxurians | Polynucleotide | SEQ ID NO: 38 |
| | | cDNA | |
| ZmCCT10-luxurians | Zea luxurians | Polypeptide | SEQ ID NO: 39 |
| ZmCCT10-mexicana | Zea mays spp mexicana | Polynucleotide | SEQ ID NO: 40 |
| | | genomic | |
| ZmCCT10-mexicana | Zea mays spp mexicana | Polynucleotide | SEQ ID NO: 41 |
| | | cDNA | |
| ZmCCT10-mexicana | Zea mays spp mexicana | Polypeptide | SEQ ID NO: 42 |
| ZmCCT10- | Zea mays spp | Polynucleotide | SEQ ID NO: 43 |
| huehuetenangensis | huehuetenangensis | genomic | |
| ZmCCT10- | Zea mays spp | Polynucleotide | SEQ ID NO: 44 |
| huehuetenangensis | huehuetenangensis | cDNA | |
| ZmCCT10- | Zea mays spp | Polypeptide | SEQ ID NO: 45 |
| huehuetenangensis | huehuetenangensis | | |
| OsGhd1 | Oryza sativa | Polynucleotide | SEQ ID NO: 46 |
| | | cDNA | |
| OsGhd1 | Oryza sativa | Polypeptide | SEQ ID NO: 47 |
| OsCCT10 | Oryza sativa | Polynucleotide | SEQ ID NO: 48 |
| | | cDNA | |
| OsCCT10 | Oryza sativa | Polypeptide | SEQ ID NO: 49 |

### Construction of Nucleic Acids

The isolated nucleic acids of the present disclosure can be made using: (a) standard recombinant methods, (b) synthetic techniques or combinations thereof. In some embodiments, the polynucleotides of the present disclosure will be cloned, amplified or otherwise constructed from a fungus or bacteria.

The nucleic acids may conveniently comprise sequences in addition to a polynucleotide of the present disclosure. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present disclosure. For example, a hexa-histidine marker sequence provides a convenient means to purify the proteins of the present disclosure. The nucleic acid of the present disclosure, excluding the polynucleotide sequence, is optionally a vector, adapter or linker for cloning and/or expression of a polynucleotide of the present disclosure. Additional sequences may be added to such cloning and/or expression sequences to optimize their function in cloning and/or expression, to aid in isolation of the polynucleotide or to improve the introduction of the polynucleotide into a cell. Typically, the length of a nucleic acid of the present disclosure less the length of its polynucleotide of the present disclosure is less than 20 kilobase pairs, often less than 15 kb and frequently less than 10 kb. Use of cloning vectors, expression vectors, adapters and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSIox and lambda MOSEIox. Optional vectors for the present disclosure, include but are not limited to, lambda ZAP II and pGEX. For a description of various nucleic acids see, e.g., Stratagene Cloning Systems, Catalogs 1995, 1996, 1997 (La Jolla, Calif.) and, Amersham Life Sciences, Inc, Catalog '97 (Arlington Heights, Ill.).

### Synthetic Methods for Constructing Nucleic Acids

The isolated nucleic acids of the present disclosure can also be prepared by direct chemical synthesis by methods such as the phosphotriester method of Narang, et al., (1979) Meth. EnzymoL 68:90-9; the phosphodiester method of Brown, et al., (1979) Meth. EnzymoL 68:109-51; the diethylphosphoramidite method of Beaucage et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395; or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include: (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Publication Number WO 1996/30530; GOS2 (U.S. Pat. No. 6,504,083) and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters are referred to here as “inducible” promoters (Rab17, RAD29). Environmental conditions that may affect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119 and hereby incorporated by reference) or signal peptides which target proteins to the plastids such as that of rapeseed enoyl-Acp reductase (Verwaert, et al., (1994) Plant Mol. Biol. 26:189-202) are useful in the disclosure. The barley alpha amylase signal sequence fused to the CCT polynucleotide is the preferred construct for expression in maize for the present disclosure.

The vector comprising the sequences from a polynucleotide of the present disclosure will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene) or other such genes known in the art. The bar gene encodes resistance to the herbicide basta, and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

### Expression in Eukaryotes

A variety of eukaryotic expression systems such as yeast, insect cell lines, plant and mammalian cells, are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) METHODS IN YEAST GENETICS, Cold Spring Harbor Laboratory is a well recognized work describing the various methods available to produce the protein in yeast. Two widely utilized yeasts for production of eukaryotic proteins are Saccharomyces cerevisiae and Pichia pastoris.Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present disclosure can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present disclosure are available, for instance, from the American Type Culture Collection Catalogue of Cell Lines and Hybridomas (7^{th} ed., 1992).

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA CLONING: A PRACTICAL APPROACH, vol. II, Glover, ed., IRL Press, Arlington, Va., pp. 213-38 (1985)).

In addition, the gene for CCT expression placed in the appropriate plant expression vector can be used to transform plant cells. The polypeptide can then be isolated from plant callus or the transformed cells can be used to regenerate transgenic plants. Such transgenic plants can be harvested and the appropriate tissues (seed or leaves, for example) can be subjected to large scale protein extraction and purification techniques.

### Plant Transformation Methods

Numerous methods for introducing foreign genes into plants are known and can be used to insert a CCT polynucleotide into a plant host, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods eds. Gamborg and Phillips, Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209; Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion or via a binary system where the virgene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Monocot plants can now be transformed with some success. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, Theor. Appl. Genet. 69:235-40 (1985); U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Increasing the Activity and/or Level of a CCT Polypeptide Encoded by CCT Polynucleotides

Methods are provided to increase the activity and/or level of the CCT polypeptides encoded by the CCT polynucleotides of the disclosure. An increase in the level and/or activity of the CCT polypeptide of the disclosure can be achieved by providing to the plant a CCT polypeptide. The CCT polypeptide can be provided by introducing the amino acid sequence encoding the CCT polypeptide into the plant, introducing into the plant a nucleotide sequence encoding a CCT polypeptide or alternatively by modifying a genomic locus encoding the CCT polypeptide of the disclosure.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having cell number regulator activity. It is also recognized that the methods of the disclosure may employ a polynucleotide that is not capable of directing, in the transformed plant, the expression of a protein or an RNA. Thus, the level and/or activity of a CCT polypeptide may be increased by altering the gene encoding the CCT polypeptide or its promoter. See, e.g., Kmiec, U.S. Pat. No. 5,565,350; Zarling, et al., PCT/US93/03868. Therefore mutagenized plants that carry mutations in CCT genes, where the mutations increase expression of the CCT gene or increase the plant growth and/or organ development activity of the encoded CCT polypeptide are provided.

Reducing the Activity and/or Level of a CCT Polypeptide

Methods are provided to reduce or eliminate the activity of a CCT polypeptide of the disclosure by transforming a plant cell with an expression cassette that expresses a polynucleotide that inhibits the expression of the CCT polypeptide. The polynucleotide may inhibit the expression of the CCT polypeptide directly, by preventing translation of the CCT messenger RNA, or indirectly, by encoding a polypeptide that inhibits the transcription or translation of a CCT gene encoding a CCT polypeptide. Methods for inhibiting or eliminating the expression of a gene in a plant are well known in the art, and any such method may be used in the present disclosure to inhibit the expression of a CCT polypeptide.

In accordance with the present disclosure, the expression of a CCT polypeptide is inhibited if the protein level of the CCT polypeptide is less than 70% of the protein level of the same CCT polypeptide in a plant that has not been genetically modified or mutagenized to inhibit the expression of that CCT polypeptide. In particular embodiments of the disclosure, the protein level of the CCT polypeptide in a modified plant according to the disclosure is less than 60%, less than 50%, less than 40%, less than 30%, less than 20%, less than 10%, less than 5% or less than 2% of the protein level of the same CCT polypeptide in a plant that is not a mutant or that has not been genetically modified to inhibit the expression of that CCT polypeptide. The expression level of the CCT polypeptide may be measured directly, for example, by assaying for the level of CCT polypeptide expressed in the plant cell or plant, or indirectly, for example, by measuring the plant growth and/or organ development activity of the CCT polypeptide in the plant cell or plant, or by measuring the biomass in the plant. Methods for performing such assays are described elsewhere herein.

In other embodiments of the disclosure, the activity of the CCT polypeptides is reduced or eliminated by transforming a plant cell with an expression cassette comprising a polynucleotide encoding a polypeptide that inhibits the activity of a CCT polypeptide. The plant growth and/or organ development activity of a CCT polypeptide is inhibited according to the present disclosure if the plant growth and/or organ development activity of the CCT polypeptide is less than 70% of the plant growth and/or organ development activity of the same CCT polypeptide in a plant that has not been modified to inhibit the plant growth and/or organ development activity of that CCT polypeptide. In particular embodiments of the disclosure, the plant growth and/or organ development activity of the CCT polypeptide in a modified plant according to the disclosure is less than 60%, less than 50%, less than 40%, less than 30%, less than 20%, less than 10% or less than 5% of the plant growth and/or organ development activity of the same CCT polypeptide in a plant that that has not been modified to inhibit the expression of that CCT polypeptide. The plant growth and/or organ development activity of a CCT polypeptide is “eliminated” according to the disclosure when it is not detectable by the assay methods described elsewhere herein. Methods of determining the plant growth and/or organ development activity of a CCT polypeptide are described elsewhere herein.

In other embodiments, the activity of a CCT polypeptide may be reduced or eliminated by disrupting the gene encoding the CCT polypeptide. The disclosure encompasses mutagenized plants that carry mutations in CCT genes, where the mutations reduce expression of the CCT gene or inhibit the plant growth and/or organ development activity of the encoded CCT polypeptide.

Thus, many methods may be used to reduce or eliminate the activity of a CCT polypeptide. In addition, more than one method may be used to reduce the activity of a single CCT polypeptide. Non-limiting examples of methods of reducing or eliminating the expression of CCT polypeptides are given below.

1. Polynucleotide-Based Methods:

In some embodiments of the present disclosure, a plant is transformed with an expression cassette that is capable of expressing a polynucleotide that inhibits the expression of a CCT polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one CCT polypeptide is an expression cassette capable of producing an RNA molecule that inhibits the transcription and/or translation of at least one CCT polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a CCT polypeptide are given below.

i. Sense Suppression/Cosuppression

In some embodiments of the disclosure, inhibition of the expression of a CCT polypeptide may be obtained by sense suppression or cosuppression. For cosuppression, an expression cassette is designed to express an RNA molecule corresponding to all or part of a messenger RNA encoding a CCT polypeptide in the “sense” orientation. Over expression of the RNA molecule can result in reduced expression of the native gene. Accordingly, multiple plant lines transformed with the cosuppression expression cassette are screened to identify those that show the greatest inhibition of CCT polypeptide expression.

The polynucleotide used for cosuppression may correspond to all or part of the sequence encoding the CCT polypeptide, all or part of the 5′ and/or 3′ untranslated region of a CCT polypeptide transcript or all or part of both the coding sequence and the untranslated regions of a transcript encoding a CCT polypeptide. In some embodiments where the polynucleotide comprises all or part of the coding region for the CCT polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

ii. Antisense Suppression

In some embodiments of the disclosure, inhibition of the expression of the CCT polypeptide may be obtained by antisense suppression. For antisense suppression, the expression cassette is designed to express an RNA molecule complementary to all or part of a messenger RNA encoding the CCT polypeptide. Over expression of the antisense RNA molecule can result in reduced expression of the native gene. Accordingly, multiple plant lines transformed with the antisense suppression expression cassette are screened to identify those that show the greatest inhibition of CCT polypeptide expression.

The polynucleotide for use in antisense suppression may correspond to all or part of the complement of the sequence encoding the CCT polypeptide, all or part of the complement of the 5′ and/or 3′ untranslated region of the CCT transcript or all or part of the complement of both the coding sequence and the untranslated regions of a transcript encoding the CCT polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a CCT polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of CCT polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of one or a CCT polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., shows 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the CCT polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the CCT polypeptide. Thus, the polynucleotide causes the degradation of the endogenous messenger RNA, resulting in reduced expression of the CCT polypeptide. This method is described, for example, in U.S. Pat. No. 4,987,071, herein incorporated by reference.

vii. Small Interfering RNA or Micro RNA

In some embodiments of the disclosure, inhibition of the expression of a CCT polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of CCT expression, the 22-nucleotide sequence is selected from a CCT transcript sequence and contains 22 nucleotides of said CCT sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

2. Polypeptide-Based Inhibition of Gene Expression

In one embodiment, the polynucleotide encodes a zinc finger protein that binds to a gene encoding a CCT polypeptide, resulting in reduced expression of the gene. In particular embodiments, the zinc finger protein binds to a regulatory region of a CCT gene. In other embodiments, the zinc finger protein binds to a messenger RNA encoding a CCT polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355, each of which is herein incorporated by reference.

3. Polypeptide-Based Inhibition of Protein Activity

In some embodiments of the disclosure, the polynucleotide encodes an antibody that binds to at least one CCT polypeptide and reduces the activity of the CCT polypeptide. In another embodiment, the binding of the antibody results in increased turnover of the antibody-CCT complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present disclosure, the activity of a CCT polypeptide is reduced or eliminated by disrupting the gene encoding the CCT polypeptide. The gene encoding the CCT polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced cell number regulator activity.

i. Transposon Tagging

In one embodiment of the disclosure, transposon tagging is used to reduce or eliminate the CCT activity of one or more CCT polypeptide. Transposon tagging comprises inserting a transposon within an endogenous CCT gene to reduce or eliminate expression of the CCT polypeptide. “CCT gene” is intended to mean the gene that encodes a CCT polypeptide according to the disclosure.

In this embodiment, the expression of one or more CCT polypeptide is reduced or eliminated by inserting a transposon within a regulatory region or coding region of the gene encoding the CCT polypeptide. A transposon that is within an exon, intron, 5′ or 3′ untranslated sequence, a promoter or any other regulatory sequence of a CCT gene may be used to reduce or eliminate the expression and/or activity of the encoded CCT polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function of the encoded protein are well known in the art. Insertional mutations in gene exons usually result in null-mutants. Mutations in conserved residues are particularly effective in inhibiting the cell number regulator activity of the encoded protein. Conserved residues of plant CCT polypeptides suitable for mutagenesis with the goal to eliminate cell number regulator activity have been described. Such mutants can be isolated according to well-known procedures, and mutations in different CCT loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more CCT polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

Modulating Plant Growth and/or Organ Development Activity

In specific methods, the level and/or activity of tissue development in a plant is increased by increasing the level or activity of the CCT polypeptide in the plant. Methods for increasing the level and/or activity of CCT polypeptides in a plant are discussed elsewhere herein. Briefly, such methods comprise providing a CCT polypeptide of the disclosure to a plant and thereby increasing the level and/or activity of the CCT polypeptide. In other embodiments, a CCT nucleotide sequence encoding a CCT polypeptide can be provided by introducing into the plant a polynucleotide comprising a CCT nucleotide sequence of the disclosure, expressing the CCT sequence, increasing the activity of the CCT polypeptide and thereby increasing the number of tissue cells in the plant or plant part. In other embodiments, the CCT nucleotide construct introduced into the plant is stably incorporated into the genome of the plant.

In other methods, the number of cells and biomass of a plant tissue is increased by increasing the level and/or activity of the CCT polypeptide in the plant. Such methods are disclosed in detail elsewhere herein. In one such method, a CCT nucleotide sequence is introduced into the plant and expression of said CCT nucleotide sequence decreases the activity of the CCT polypeptide and thereby increasing the plant growth and/or organ development in the plant or plant part. In other embodiments, the CCT nucleotide construct introduced into the plant is stably incorporated into the genome of the plant.

As discussed above, one of skill will recognize the appropriate promoter to use to modulate the level/activity of a plant growth and/or organ development polynucleotide and polypeptide in the plant. Exemplary promoters for this embodiment have been disclosed elsewhere herein.

Accordingly, the present disclosure further provides plants having a modified plant growth and/or organ development when compared to the plant growth and/or organ development of a control plant tissue. In one embodiment, the plant of the disclosure has an increased level/activity of the CCT polypeptide of the disclosure and thus has increased plant growth and/or organ development in the plant tissue. In other embodiments, the plant of the disclosure has a reduced or eliminated level of the CCT polypeptide of the disclosure and thus has decreased plant growth and/or organ development in the plant tissue. In other embodiments, such plants have stably incorporated into their genome a nucleic acid molecule comprising a CCT nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the CCT polypeptide in the plant. In one method, a CCT sequence of the disclosure is provided to the plant. In another method, the CCT nucleotide sequence is provided by introducing into the plant a polynucleotide comprising a CCT nucleotide sequence of the disclosure, expressing the CCT sequence and thereby modifying root development. In still other methods, the CCT nucleotide construct introduced into the plant is stably incorporated into the genome of the plant.

In other methods, root development is modulated by altering the level or activity of the CCT polypeptide in the plant. An increase in CCT activity can result in at least one or more of the following alterations to root development, including, but not limited to, larger root meristems, increased in root growth, enhanced radial expansion, an enhanced vasculature system, increased root branching, more adventitious roots and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by increasing the activity and/or level of the CCT polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by increasing the level and/or activity of the CCT polypeptide also finds use in promoting in vitro propagation of explants.

Furthermore, higher root biomass production due to an increased level and/or activity of CCT activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of the CCT polypeptide of the disclosure and has enhanced root growth and/or root biomass. In other embodiments, such plants have stably incorporated into their genome a nucleic acid molecule comprising a CCT nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a CCT polypeptide of the disclosure. In one embodiment, a CCT sequence of the disclosure is provided. In other embodiments, the CCT nucleotide sequence can be provided by introducing into the plant a polynucleotide comprising a CCT nucleotide sequence of the disclosure, expressing the CCT sequence and thereby modifying shoot and/or leaf development. In other embodiments, the CCT nucleotide construct introduced into the plant is stably incorporated into the genome of the plant.

In specific embodiments, shoot or leaf development is modulated by decreasing the level and/or activity of the CCT polypeptide in the plant. A decrease in CCT activity can result in at least one or more of the following alterations in shoot and/or leaf development, including, but not limited to, reduced leaf number, reduced leaf surface, reduced vascular, shorter internodes and stunted growth and retarded leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Decreasing CCT activity and/or level in a plant results in shorter internodes and stunted growth. Thus, the methods of the disclosure find use in producing dwarf plants. In addition, as discussed above, modulation of CCT activity in the plant modulates both root and shoot growth. Thus, the present disclosure further provides methods for altering the root/shoot ratio. Shoot or leaf development can further be modulated by decreasing the level and/or activity of the CCT polypeptide in the plant.

Accordingly, the present disclosure further provides plants having modulated shoot and/or leaf development when compared to a control plant. In some embodiments, the plant of the disclosure has an increased level/activity of the CCT polypeptide of the disclosure, altering the shoot and/or leaf development. Such alterations include, but are not limited to, increased leaf number, increased leaf surface, increased vascularity, longer internodes and increased plant stature, as well as alterations in leaf senescence, as compared to a control plant. In other embodiments, the plant of the disclosure has a decreased level/activity of the CCT polypeptide of the disclosure.

vi Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the CCT polypeptide has not been modulated. “Modulating floral development” further includes any alteration in the timing of the development of a plant's reproductive tissue (i.e., a delayed or an accelerated timing of floral development) when compared to a control plant in which the activity or level of the CCT polypeptide has not been modulated. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating CCT activity in a plant. In one method, a CCT sequence of the disclosure is provided. A CCT nucleotide sequence can be provided by introducing into the plant a polynucleotide comprising a CCT nucleotide sequence of the disclosure, expressing the CCT sequence and thereby modifying floral development. In other embodiments, the CCT nucleotide construct introduced into the plant is stably incorporated into the genome of the plant.

In specific methods, floral development is modulated by decreasing the level or activity of the CCT polypeptide in the plant. A decrease in CCT activity can result in at least one or more of the following alterations in floral development, including, but not limited to, retarded flowering, reduced number of flowers, partial male sterility and reduced seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by increasing the level and/or activity of the CCT sequence of the disclosure. Such methods can comprise introducing a CCT nucleotide sequence into the plant and increasing the activity of the CCT polypeptide. In other methods, the CCT nucleotide construct introduced into the plant is stably incorporated into the genome of the plant. Increasing expression of the CCT sequence of the disclosure can modulate floral development during periods of stress. Such methods are described elsewhere herein. Accordingly, the present disclosure further provides plants having modulated floral development when compared to the floral development of a control plant. Compositions include plants having an increased level/activity of the CCT polypeptide of the disclosure and having an altered floral development. Compositions also include plants having an increased level/activity of the CCT polypeptide of the disclosure wherein the plant maintains or proceeds through the flowering process in times of stress.

Methods are also provided for the use of the CCT sequences of the disclosure to increase seed size and/or weight. The method comprises increasing the activity of the CCT sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for decreasing seed size and/or seed weight in a plant comprises decreasing CCT activity in the plant. In one embodiment, the CCT nucleotide sequence can be provided by introducing into the plant a polynucleotide comprising a CCT nucleotide sequence of the disclosure, expressing the CCT sequence and thereby decreasing seed weight and/or size. In other embodiments, the CCT nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has an increased level/activity of the CCT polypeptide of the disclosure and has an increased seed weight and/or seed size. In other embodiments, such plants have stably incorporated into their genome a nucleic acid molecule comprising a CCT nucleotide sequence of the disclosure operably linked to a promoter that drives expression in the plant cell.

Method of Use for CCT Promoter Polynucleotides

The polynucleotides comprising the CCT promoters disclosed in the present disclosure, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the CCT promoter polynucleotides of the disclosure are provided in expression cassettes along with a polynucleotide sequence of interest for expression in the host cell of interest. As discussed in the Examples section of the disclosure, the CCT promoter sequences of the disclosure are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the disclosure, heterologous sequence expression is controlled by a synthetic hybrid promoter comprising the CCT promoter sequences of the disclosure, or a variant or fragment thereof, operably linked to upstream promoter element(s) from a heterologous promoter. Upstream promoter elements that are involved in the plant defense system have been identified and may be used to generate a synthetic promoter. See, for example, Rushton, et al., (1998) Curr. Opin. Plant Biol. 1:311-315. Alternatively, a synthetic CCT promoter sequence may comprise duplications of the upstream promoter elements found within the CCT promoter sequences.

It is recognized that the promoter sequence of the disclosure may be used with its native CCT coding sequences. A DNA construct comprising the CCT promoter operably linked with its native CCT gene may be used to transform any plant of interest to bring about a desired phenotypic change, such as modulating cell number, modulating root, shoot, leaf, floral and embryo development, stress tolerance and any other phenotype described elsewhere herein.

The promoter nucleotide sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading, and the like.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene) and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996 and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502, herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production or provide expression of proteins. Another important commercial use of transformed plants is the production of polymers and bioplastics such as described in U.S. Pat. No. 5,602,321. Genes such as 13-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

viii. Identification of Additional Cis-Acting Elements

Additional cis-elements for the CCT promoters disclosed herein can be identified by a number of standard techniques, including for example, nucleotide deletion analysis, i.e., deleting one or more nucleotides from the 5′ end or internal to a promoter and assaying for regulatory activity, DNA binding protein analysis using DNase I footprinting, methylation interference, electrophoresis mobility-shift assays, in vivo genomic footprinting by ligation-mediated PCR and other conventional assays or by DNA sequence similarity analysis with other known cis-element motifs by conventional DNA sequence comparison methods and by statistical methods such as hidden Markov model (HMM). cis-elements can be further analyzed by mutational analysis of one or more nucleotides or by other conventional methods.

ix. Chimeric Promoters

Chimeric promoters that combine one or more cis-elements are known (see, Venter, et al., (2008), Trends in Plant Science, 12(3):118-124). Chimeric promoters that contain cis-elements from the promoters disclosed herein along with their flanking sequences can be engineered into other promoters that are for example, tissue specific. For example, a chimeric promoter may be generated by fusing a first promoter fragment containing the activator (CCT) cis-element from one promoter to a second promoter fragment containing the activator (tissue-specific) cis-element from another promoter; the resultant chimeric promoter may increase gene expression of the linked transcribable polynucleotide molecule in both CCT and tissue specific manner. Regulatory elements disclosed herein are used to engineer chimeric promoters, for example, by placing such an element upstream of a minimal promoter.

This disclosure can be better understood by reference to the following non-limiting examples. It will be appreciated by those skilled in the art that other embodiments of the disclosure may be practiced without departing from the spirit and the scope of the disclosure as herein disclosed and claimed.

### EXAMPLES

### Example 1

### Identification and Cloning of the Maize Photoperiod Regulator ZmCCT10 Gene

The current genetic research of maize photoperiod sensitivity is based on QTL (Quantitative Trait Locus) mapping for flowering time. Four major photoperiod sensitivity loci were identified (Genetics (2010) 184:799-812) and a major QTL was mapped to chromosome 10 (Genetics 183:1555-1563). The QTL on chromosome 10 is mapped to the CCT domain gene (Proc Natl Acad Sci USA (2012) 109(28)). The CCT domain (stands for three founder genes CONSTANS, CONSTANS-LIKE, TOC1) is a carboxyl-terminus ˜45 amino acid peptide, rich in basic residues, and contains a putative nuclear localization signal (Science (2000) 289(5480):768-71). The CCT domains typically are present in proteins involved in transmission of light signals.

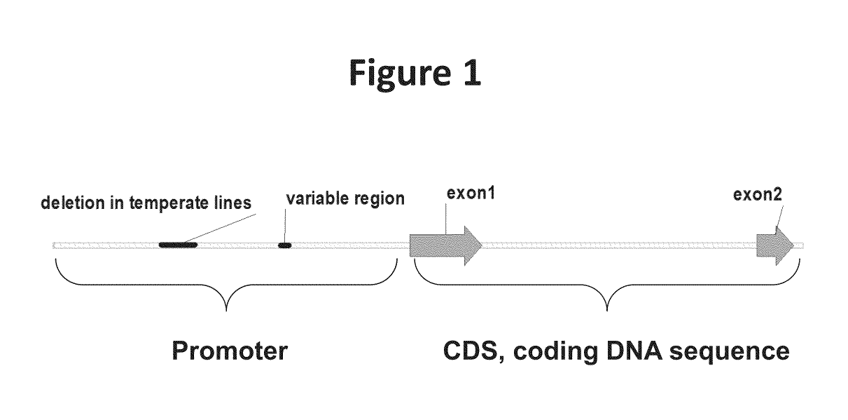

To identify a gene corresponding to the photoperiod sensitivity QTL on chromosome 10, a number of primers (Table 1) were designed to amplify a gene by PCR (polymerase chain reaction). Six maize lines with different photoperiod sensitivity were used for DNA amplification: early flowering day-neutral line Gaspé flint; temperate flowering day-neutral B73 inbred; short-day tropical inbred DCBNF, BC26N, CML436, maize wild progenitors Zm-huehuetenangensis, Zm-mexicana, Zm-parviglumis and Zea luxurians known under the common name as teosinte. Genomic PCR fragments were cloned and sequenced (Table 1 and SEQ listing). The gene was named ZmCCT10 after Zea mays CCT chromosome 10. cDNA corresponding to the ZmCCT10 genomic copy was cloned by RT-PCR using RNA isolated from leaves grown under the long days when ZmCCT10 expression reaches its maximum. Alignment of genomic and cDNA revealed the gene structure which is composed of two exons and the 2.3-kb promoter region (FIG. 1).

### Example 2

### Identification and Cloning of the Sorghum Photoperiod Regulators SbCCT1 and SbCCT6 Genes

Sorghum is a short-day plant originated in northern Africa. Similar to maize, sorghum is photoperiod sensitive and cultivated widely in tropical and subtropical regions. To identify the sorghum CCT homologous genes “Sorghum Predicted Coding Sequences (JGI, Joint Genome Institute v.1.4) (N)” were searched by BLAST using ZmCCT10 and rice Ghd7 coding nucleotide and amino acid sequence as queries. Rice Ghd7 (stands for Grain number, plant Height and heading Date 7) is a CCT gene which functions as a major negative regulator of flowering time under the non-permissive long days conditions (Itoh, et al., (2010) Nat Genet. 42(7):635-8). Two predicted coding DNA sequences were found Sb06g000570.1 and Sb01g029080.1 located to chromosome 6 and chromosome 1 respectively. These genes were named SbCCT1 and SbCCT6 which stands for Sorghum bicolor CCT chromosome 1 and chromosome 6 (JGI) The genomic sequences were retrieved from a complete sorghum genome available on the public site of Joint Genome Institute. For SbCCT1, the sorghum gene ID is Sb01g029080.1; it corresponds to Chromosome 1: 50814691 to 50817418. For SbCCT6, the sorghum gene ID is Sb06g000570.1; it corresponds to Chromosome 6: 670721 to 673363. Similar to maize ZmCCT10 gene, sorghum genes are composed of two exons.

### Example 3

### Phylogenic Analysis and Functional Domains of ZmCCT10 Predicted Polypeptide

The CCT (stands for CONSTANS, CO-like, and TOC1) domain is a highly conserved basic module of ˜43 amino acids, which is found near the C-terminus of plant proteins often involved in light signal transduction. The founder protein is encoded by the Arabidopsis CONSTANS gene which controls flowering time by linking the circadian clock with the downstream flowering genes (Suarez-Lopez, et al., (2001) Nature 410:1116-1120).

To elucidate phylogenetic relationship of the ZmCCT10 protein with other CONSTANS and CO-like plant proteins, 54 proteins were selected from different plant species including Arabidopsis, rice, maize and sorghum. A phylogenetic consensus tree was built out of bootstrap 1000.00 trees. ZmCCT10 protein formed a monophyletic group with sorghum SbCCT1 and SbCCT6 and the rice Ghd7 and OsCCT10. The Ghd7 rice QTL for grain and heading time and functions as the repressor of flowering under the long days (Itoh, et al., (2010) Nat Genet. 42:635-8). OsCCT10 is a homologous gene on chromosome 10 that was identified via the BLAST search of the rice genome, but no function is assigned to this gene.

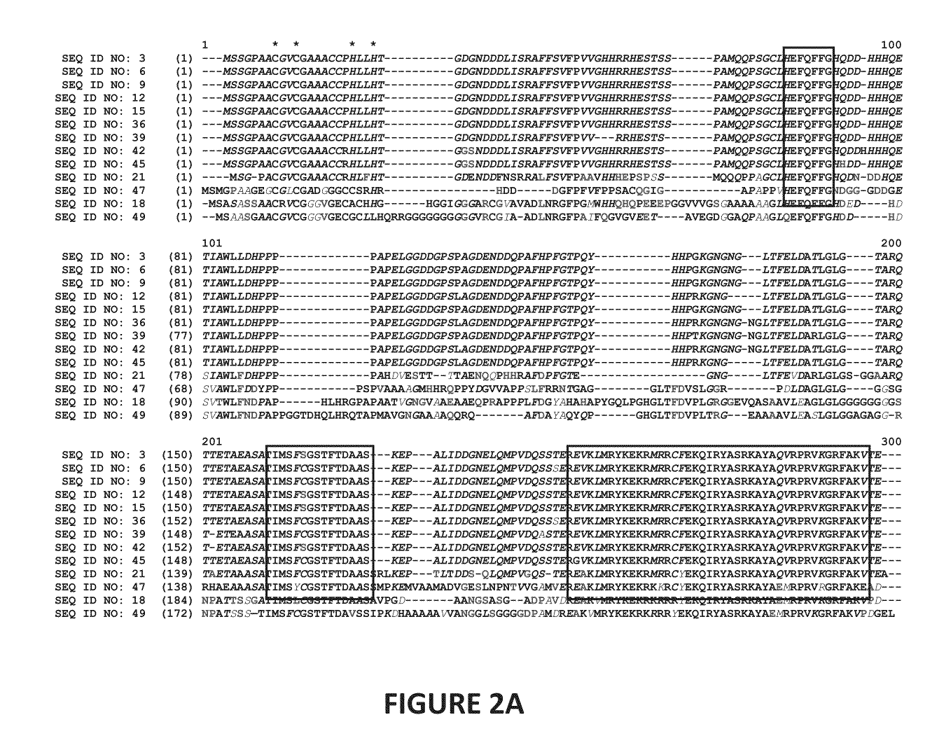

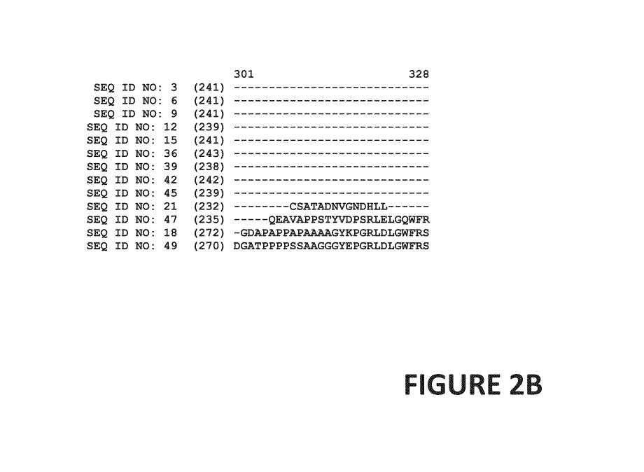

Polypeptide alignments of closely related CCT proteins from maize and subspecies, rice and sorghum revealed common motives suggesting functional relationships of these proteins. The conserved 43 amino acid CCT domain is located at C-terminus implying their common function in light transduction. The putative DNA/RNA binding motif C-X₂-C-Xₙ-H-X₂-H was identified at the N-terminus (FIG. 2). This motif may be described as the zinc finger-CCCH type suggesting that these proteins has a potential binding to nucleic acids and function as the transcriptional regulators.

### Example 4

### Expression of ZmCCT10 in Temperate and Tropical Under the Short and Long Days

Arabidopsis is sensitive to photoperiod with long days promoting flowering. The circadian clock-associated protein GIGANTEA (GI) regulates expression of CONSTANS (CO), which transcripts predominantly accumulate in the leaves under long days displaying a CCT expression pattern with the highest peak at dusk. CO activates transcription of the flowering activator FLOWERING LOCUS T (FT) gene. FT encodes a small protein which are belong the PEBP protein family which are conserved across plants and animals (Kardailsky, et al., (1999) Science 286:1962-5; Kobayashi, et al., (1999) Science 286:1960-2). FT is thought to function as the flowering hormone “florigen”. The accumulation of high levels of CO protein at the end of the day drives accumulation of FT mRNA at the end of day under long days in leaves promoting flowering under long days (Turck, et al., (2008) Annu Rev Plant Biol 59:573-594).

Rice is sensitive to photoperiod with short days promoting flowering. The GI-CO-FT pathway is conserved in rice but regulatory modifications of this module are significantly diverged from Arabidopsis (Tsuji, et al., (2010) Curr Opin Plant Biol. 14:1-8). There are two “florigen” genes in rice both of them encoded FT homologous proteins. Hd3a and its paralog RFT1 are located on chromosome 6 approximately 10 kb apart. Hd3a works as the floral activator under inductive short days whereas RFT1 is the activator under non-inductive long days.

Under the inductive short days Hd3a is activated by Hd1, the rice CO ortholog, and by the rice-specific B-type response regulator Ehd1. Under non-inductive long days Hd1 works as a repressor of Hd3a. More over the Ehd1 activator is suppressed by the Ghd7 protein, which is a small protein with a CCT domain. Ghd7 expression is specifically up-regulated during long days and subsequently suppresses Ehd1-Hd3a expression. Nevertheless rice can finally flower under long days due to activity of the second florigen RFT1. When both Hd3a and RFT1 florigens are inactivated due to mutations or RNAi the rice plant never flowers. Thus both florigens are independently controlled by the short day and long day genetic pathways to allow the rice plant to adapt to different day lengths.

The temperate maize is a day neutral plant but its wild ancestor teosinte and tropical varieties are short day plants. Previously efforts identified the maize FT like florigen named ZCN8 after Zea mays CENTRORADIALIS (Danilevskaya, et al., (2008) Plant Physiol 146:250-64, Meng, et al., (2011) Plant Cell 223(3):942-60). The GI-CO-FT pathway appeared to be conserved in maize (Miller, et al., (2008) Planta. 227(6):1377-88).

ZmCCT10 was identified as a maize homolog of the rice Ghd7, which is a floral repressor under long days in rice. To be a repressor of the flowering time under the long days, one can expect ZmCCT10 to have high expression under the long day and low expression under the short days. To test this hypothesis, RT-PCR of the ZmCCT10 gene and 16 maize CO-like genes were performed under the short and long days in the temperate photoperiod insensitive maize line B73 and the tropical photoperiod sensitive CML436. Only ZmCCT10 showed reduced expression under the short days and enhanced under the long days in both temperate and tropical lines. The other CO-like genes were equally expressed under both the short and long days.

To measure precisely ZmCCT10 transcription in temperate and tropical lines, qRT-PCR was performed on leaves collected over period of three days in 4 hours intervals under short and long days. Under inductive short days the level of ZmCCT10 transcripts was very low in all genotypes tested. Conversely under non-inductive long days, the level of ZmCCT10 transcripts was slightly elevated in the temperate lines Gaspe and B73, but no rhythmic pattern was found.

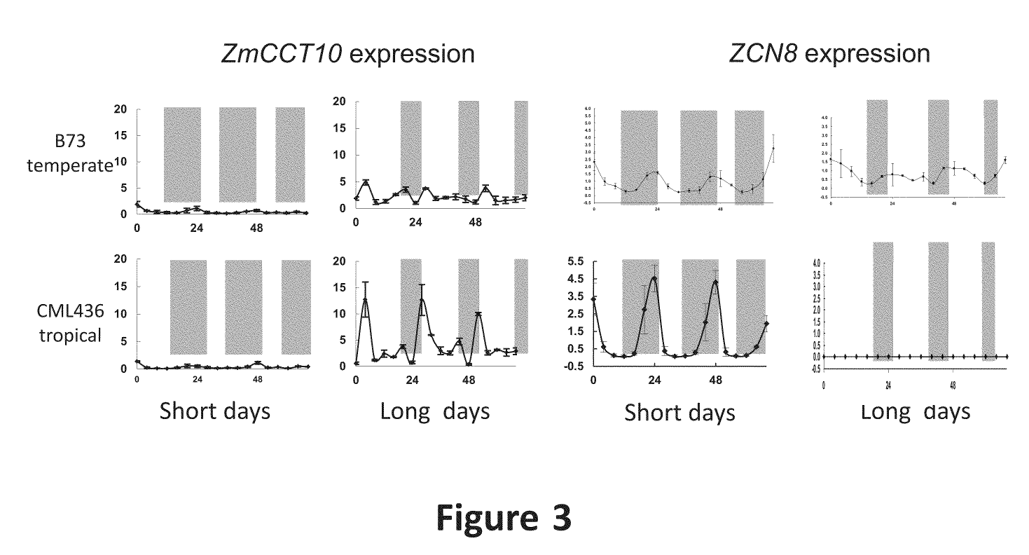

The profound distinct rhythmic transcription of ZmCCT10 was found in the tropical line CML436 with a peak of expression at 10 am (FIG. 3). If the primary target of ZmCCT10 is a floral activator ZCN8 gene, than ZCN8 expression should be in the opposite phase compared to ZmCCT10. It means that higher level of ZmCCT10 should repressed ZCN8 expression, and vise verse. The expression of the ZCN8 and ZmCCT10 gene in the same leaf samples was measured (FIG. 3).

When ZmCCT10 transcription is very low under short days in tropical line, ZCN8 expression is very high with the peak of expression just at dawn. When ZmCCT10 expression is high with the peak of expression in the morning under long days, ZCN8 transcription is very low (FIG. 3). Hence activation of ZmCCT10 just after the ZCN8 peak represses the floral activator almost to the zero. Our data support the hypothesis that ZmCCT10 is a negative regulator of the flowering time in the tropical lines under long days.

In a complete agreement with our hypothesis in the temperate day neutral corn, ZmCCT10 expression level is lower under both day lengths and subsequently ZCN8 expression is high and it promotes flowering independently of the day length (FIG. 3). Therefore ZmCCT10 was subjected to selection for low expression during corn breeding for adaptation to long days at the northern latitudes by early Indian farmers.

### Example 5

### Natural Variation in ZmCCT10 Genes in Various Maize Lines

Multiple genetic events could take place during breeding for the temperate photoperiod insensitive modern corn. ZmCCT10 would be one of the targets during selection for photoperiod insensitive corn.

To identify genetic variations associated with photoperiod insensitivity phenotypes cloning and sequencing of ZmCCT10 from temperate lines B73, Gaspe Flint, from tropical lines DCBNF, BC26N, CML436 and teosinte was performed. Six amino acid substitutions were found in the coding regions of ZmCCT10 deduced polypeptide but none of them were correlated with photoperiod sensitivity. However significant variability was found in the upstream promoter regions (FIG. 1). The 100 nucleotide deletion, located 2 kb upstream of the start ATG codon, is present in temperate lines. The variable region, located 0.6 kb upstream of the start ATG codon, showed significant insertion-deletion rearrangement between lines. It may be a hot spot for transposable elements that can change a promoter function and adjacent gene expression. It is likely that the selective pressure during corn adaptation to longer days was applied for the promoter variation in ZmCCT10 that reduced its transcriptional activity and decoupled from the circadian machinery. Genetically it could be achieved due to deletion-insertion process in the regulatory regions caused by mobility of the transposable elements. One can used these genetic variations for designing molecular markers in breeding programs using the tropical germplasm.

### Example 6

### Over-Expression of ZmCCT10 in Transgenic Plants

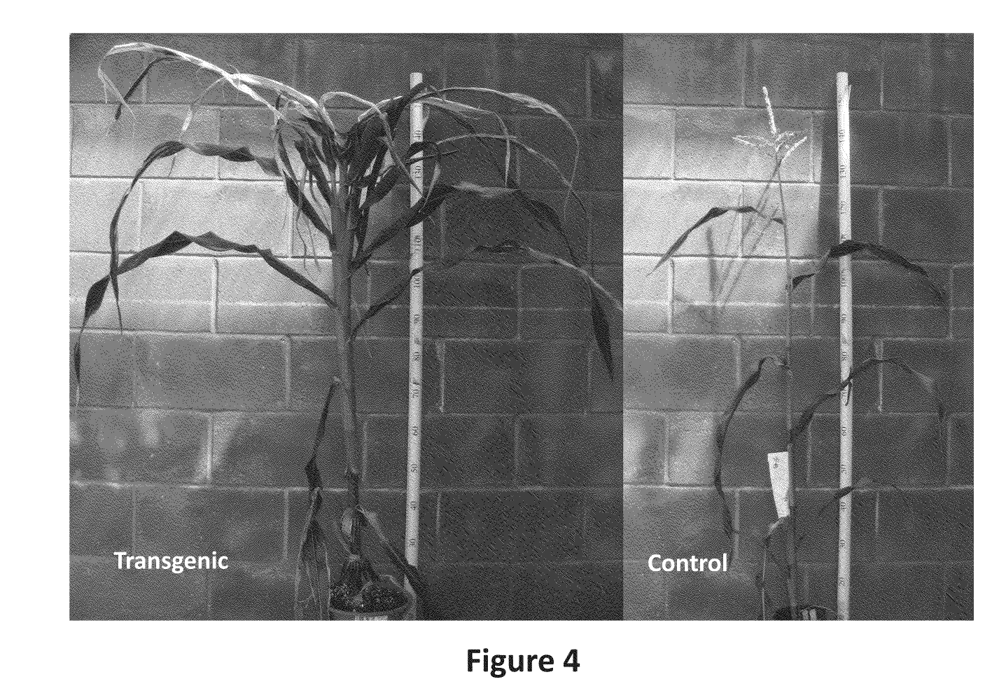

To assess function of the ZmCCT10 gene transgenic plants were generated expressing ZmCCT10 genomic DNA (SEQ ID NO: 33) driven by the constitutive ubiquitin promoter (McElroy and Brettell, (1994) Trends in Biotechnology 12:62-68.) ZmCCT10 appeared to be a potent repressor of flowering time. Its ectopic expression in the early flowering Gaspe Flint background resulted in pleiotropic phenotypes typical for tropical lines grown under the long day conditions such as the brace roots, thicker stalks, longer shanks, longer and wider leaves (FIG. 4). The further study of the T1 transgenic plants confirmed the late flowering phenotype observed in the T0 generation. The transgenic plants produced on average 12.5 leaves compared to 9.5 leaves of non-transgenic plants. Pollen shedding occurred a week later.

### Example 7

### Down-Regulation of ZmCCT10 in Transgenic Plants

The transgenic cassettes designed to down-regulate ZmCCT10 activity by means of any methods listed in “DETAILED DESCRIPTION OF THE DISCLOSURE” (Sense Suppression/Cosuppression, Antisense Suppression, Double-Stranded RNA Interference, Hairpin RNA Interference and Intron-Containing Hairpin RNA Interference, Amplicon-Mediated Interference, Ribozymes, Small Interfering RNA or Micro RNA, Polypeptide-Based Inhibition of Gene Expression, Polypeptide-Based Inhibition of Protein Activity) will be transformed in an elite background.

These cassettes will create dominant-negative alleles that will work in trans-configuration to down regulate ZmCCT10 in hybrid combinations. Because the elite lines are day-length neutral temperate corn, no obvious phenotype should be produced by the cassettes. However when crossed with day-length sensitive topical lines, the F1 hybrids should demonstrate less long-day sensitivity due to reduced ZmCCT10 function. As a result the F1 crosses of transgenic×tropical hybrids should flower earlier under the long summer days in the northern latitudes allowing breeders to conduct screening for beneficial agronomic traits (drought, disease resistance so far) without the confounding effects of flowering time.

Future gains from selection in elite temperate maize may be impeded by a lack of useful genetic diversity. Most of the genetic variation in maize is found in germplasm adapted to tropical regions near the equator. Tropical adapted maize is frequently day-length sensitive, and in long-day temperate environments, tropical material frequently delays flowering time. This impedes the introgression of tropical maize into temperate breeding programs. It may be possible to genetically down-regulate the ZmCCT10 gene in tropical maize, which may reduce photoperiod sensitivity in these lines, allowing for rapid screening and introgression of tropical alleles into elite temperate germplasm.

### Example 8

### Transformation and Regeneration of Transgenic Plants

Immature maize embryos from greenhouse donor plants are bombarded with a plasmid containing the transformation sequence operably linked to the constitutive Ubi promoter (McElroy and Brettnel, (1994) Trends Biotechology 12:62-68) and the selectable marker gene PAT, which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox@ bleach plus 0.5% Micro detergent for 20 minutes and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the transformation sequence operably linked to an ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased drought tolerance. Assays to measure improved drought tolerance are routine in the art and include, for example, increased kernel-earring capacity yields under drought conditions when compared to control maize plants under identical environmental conditions. Alternatively, the transformed plants can be monitored for a modulation in meristem development (i.e., a decrease in spikelet formation on the ear). See, for example, Bruce, et al., (2002) Journal of Experimental Botany 53:1-13.

Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/I 2,4-D and 2.88 g/l L-proline (brought to volume with D-I H₂O following adjustment to pH 5.8 with KOH); 2.0 g/l Gelrite® (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose and 2.0 mg/l 2,4-D (brought to volume with D-I H₂O following adjustment to pH 5.8 with KOH); 3.0 g/l Gelrite® (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite® (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL and 0.40 g/l glycine brought to volume with polished D-I H₂O), 0.1 g/l myo-inositol and 40.0 g/l sucrose (brought to volume with polished D-I H₂O after adjusting pH to 5.6) and 6 g/l Bacto™-agar (added after bringing to volume with polished D-I H₂O), sterilized and cooled to 60° C.

### Example 9

### Agrobacterium-Mediated Transformation

For Agrobacterium-mediated transformation of maize with an antisense sequence of the transformation sequence of the present disclosure, preferably the method of Zhao is employed (U.S. Pat. No. 5,981,840 and PCT Publication Number WO 1998/32326, the contents of which are hereby incorporated by reference). Briefly, immature embryos are isolated from maize and the embryos contacted with a suspension of Agrobacterium, where the bacteria are capable of transferring the transformation sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants. Plants are monitored and scored for a modulation in meristem development. For instance, alterations of size and appearance of the shoot and floral meristems and/or increased yields of leaves, flowers and/or fruits.

### Example 10

### Variants of Photoperiod sensitive Sequences

A. Variant Nucleotide Sequences of Photoperiod Sensitive Sequences that do not Alter the Encoded Amino Acid Sequence

The photoperiod sensitive nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding polynucleotide These functional variants are generated using a standard codon table. While the nucleotide sequence of the variants are altered, the amino acid sequence encoded by the open reading frames do not change.

B. Variant Amino Acid Sequences of Photoperiod Sensitive Polypeptides

Variant amino acid sequences of the photoperiod sensitive polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

C. Additional Variant Amino Acid Sequences of Photoperiod Sensitive Polypeptides

In this example, artificial protein sequences are created having 80%, 85%, 90% and 95% identity relative to the reference protein sequence. This latter effort requires identifying conserved and variable regions from the alignment and then the judicious application of an amino acid substitutions table. These parts will be discussed in more detail below.

Largely, the determination of which amino acid sequences are altered is made based on the conserved regions among each photoperiod sensitive protein or among the other polypeptides. Based on the sequence alignment, the various regions of the polypeptide that can likely be altered are represented in lower case letters, while the conserved regions are represented by capital letters. It is recognized that conservative substitutions can be made in the conserved regions below without altering function. In addition, one of skill will understand that functional variants of the sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 2.

TABLE 2
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the polypeptides are generating having about 80%, 85%, 90% and 95% amino acid identity to the starting unaltered ORF nucleotide sequence of SEQ ID NOS: 2, 5, 8, 11, 14, 17, 20, 33, 35, 38, 41 and 44.

### Example 11

### Alteration of Traits in Plants with the Use of Regulatory Elements and Polypeptides Disclosed Herein

The various regulatory elements including photoperiod sensitive promoters and photoperiod sensitive polypeptides disclosed herein are useful for a variety of trait development for crop plants. These include engineering freezing or frost tolerance, chilling or cold tolerance, drought or heat tolerance, salt stress tolerance, reduced photorespiration, stomatal aperture regulation, photosynthetic efficiency for yield increase, carbohydrate metabolism and transport, enhanced nitrogen utilization, selective metabolite biosynthesis, improved nutrient assimilation, source/sink modulation, disease resistance, insect resistance and pest resistance. One or more regulatory elements disclosed herein are combined with other regulatory elements including various stress inducible or tissue specific motifs to optimize transgene expression.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

1. A method of screening a population of plants for improved tolerance to biotic or abiotic stress, the method comprising:
a. providing a population of plants, wherein the photoperiod sensitivity of the plants is altered due to the down regulation of an endogenous gene, wherein the endogenous gene encodes a protein that comprises a CONSTANS, CONSTANS-LIKE, TOC1 (CCT) domain; and
b. screening the population of plants for the improved tolerance to biotic or abiotic stress.

2. The method of claim 1, wherein the population of plants with reduced CCT gene expression is grown in a plant growing environment that is different than a normal growing environment for the population of plants in the absence of the down regulation of the endogenous gene.

3. The method of claim 1, wherein the endogenous gene encodes a polypeptide that comprises a CCT domain that is at least 90% identical to the CCT domain of SEQ ID NO: 3.

4. (canceled)

5. (canceled)

6. (canceled)

7. (canceled)

8. (canceled)

9. (canceled)

10. (canceled)

11. (canceled)

12. (canceled)

13. A method of modulating photoperiod sensitivity in a plant, comprising:
a. providing a photoperiod sensitive plant with a recombinant polynucleotide of claim 2, the polynucleotide operably linked to a promoter, wherein the polynucleotide down regulates the expression of an endogenous gene encoding a polypeptide comprising a CONSTANS, CONSTANS-LIKE, TOC1 (CCT) domain;
b. reducing the photoperiod sensitivity of the plant by downregulating the expression endogenous gene.

14. The method of claim 13, wherein the plant is from a plant selected from the group consisting of: maize, soybean, sunflower, sorghum, canola, wheat, alfalfa, cotton, rice, barley, millet, peanut, sugar cane and cocoa.

15. The method of claim 1, wherein the plant is selected from the group consisting of:
maize, soybean, sorghum, canola, wheat, alfalfa, cotton, rice, barley, millet, peanut and cocoa.

16. A recombinant expression cassette, comprising a polynucleotide selected from:
a. a polynucleotide having at least 90% sequence identity, as determined by the GAP algorithm under default parameters, to the full length sequence of a polynucleotide selected from the group consisting of SEQ ID NOS: 2, 5, 8, 11, 14, 17, 20, 33, 35, 38, 41 and 44; wherein the polynucleotide encodes a polypeptide that modifies photoperiod sensitivity;
b. a polynucleotide selected from the group consisting of SEQ ID NOS: 2, 5, 8, 11, 14, 17, 33, 35, 38, 41 and 44;
c. a polynucleotide which is fully complementary to the polynucleotide of (a) or (b);
d. a polypeptide encoded by the polynucleotide of (a) or (b); and
e. a polypeptide having at least 90% sequence identity, as determined by the GAP algorithm under default parameters, to the full length sequence of a polypeptide selected from the group consisting of SEQ ID NOS: 2, 5, 8, 11, 14, 17, 20, 33, 35, 38, 41 and 44;
wherein the polynucleotide is operably linked, in sense or anti sense orientation, to a promoter.

17. A host cell comprising the expression cassette of claim 16.

18. A transgenic plant comprising the recombinant expression cassette of claim 16.

19. (canceled)

20. (canceled)

21. The transgenic plant of claim 18, wherein said plant is selected from the group consisting of: maize, soybean, sunflower, sorghum, canola, wheat, alfalfa, cotton, rice, barley, millet, peanut, sugar cane and cocoa.

22. A transgenic seed from the transgenic plant of claim 18.

23. A recombinant expression cassette of claim 13 comprising a regulatory polynucleotide molecule comprising a sequence selected from the group consisting of: (a) SEQ ID NOS: 1, 4, 7, 10, 13, 16, 19, 33, 34, 37, 40, and 43; (b) a nucleic acid fragment that comprises at least 50-100 contiguous nucleotides of one of SEQ ID NOS: 1, 4, 7, 10, 13, 16, 19, 33, 34, 37, 40, and 43 and wherein the fragment comprises one or more of the photoperiod sensitive sequences listed in Table 1 and (c) a nucleic acid sequence comprising at least 90% identity to about 500-1000 contiguous nucleotides of one of SEQ ID NOS: 1, 4, 7, 10, 13, 16, 19 33, 34, 37, 40, and 43 as determined by the GAP algorithm under default parameters.

24. A chimeric polynucleotide molecule comprising the nucleic acid fragment of claim 23.

25. The regulatory polynucleotide molecule of claim 23, wherein said regulatory polynucleotide molecule is a promoter.

26. A construct comprising the regulatory molecule of claim 23 operably linked to a heterologous polynucleotide molecule, wherein the heterologous molecule confers a trait of interest.

27. The construct of claim 26, wherein the trait of interest is selected from the group consisting of drought tolerance, freezing tolerance, chilling or cold tolerance, disease resistance and insect resistance.

28. (canceled)

29. A transgenic plant transformed with the expression cassette of claim 23.

30. (canceled)

31. The transgenic plant of claim 30 is selected from the group consisting of maize, soybean, canola, cotton, sunflower, alfalfa, sugar beet, wheat, rye, rice, sugarcane, oat, barley, turf grass, sorghum, millet, tomato, pigeon pea, vegetable, fruit tree and forage grass.

32. (canceled)

33. (canceled)

34. A method of decreasing the photoperiod sensitivity in a plant, comprising:
(a) providing a polynucleotide having a nucleotide sequence comprising at least 15 consecutive nucleotides of the complement of SEQ ID NOS: 2, 5, 8, 11, 14, 17, 20, 33, 35, 38, 41 or 44;
(b) providing a plant comprising a mRNA having the sequence set forth in SEQ ID NOS: 2, 5, 8, 11, 14, 17, 20, 33, 35, 38, 41 or 44; and
(c) introducing the polynucleotide of step (a) into the plant, wherein the nucleotide sequence inhibits expression of the mRNA in the plant and the plant has decreased photoperiod sensitivity compared with a plant that does not comprise the polynucleotide of step (a).

35. (canceled)

36. (canceled)

37. (canceled)

38. (canceled)

39. (canceled)

40. A method of identifying alleles in corn plants or corn germplasm associated with altered photoperiod sensitivity, the method comprising:
a. obtaining a population of corn plants, wherein one or more plants exhibit altered photoperiod sensitivity;
b. evaluating allelic variations with respect to the polynucleotide sequence encoding a protein comprising a polypeptide:
i. encoded by the expression cassette of claim 16;
c. obtaining phenotypic values of altered photoperiod sensitivity for a plurality of corn plants in the population;
d. associating the allelic variations in the genomic region associated with the polynucleotide with the phenotype; and
e. identifying the alleles that are associated with altered photoperiod sensitivity.


SEQUENCE LISTING (49 sequences)

SEQ ID NO: 1 - DNA, 5219 bp, Zea mays
gctatagctttgttaaaaggcagcagcaaaattcgaagctggggccagcggccattgattcctgatggatgcacatacgtgcatccatatcgatgctgtcgctgcctccgtcgtctcgtctcgccacaaccgcgctacacgcaaaagctatgcggattctgtttctgtgtaaacaaccaacactctctttattccaatggacaatgctccatctctcttgtgccccccgcaggacagaattggctaggcggcgattccacgtgccatcacatttccaattctgaagttagcctatcccaccgttgcttcctacacgtgtcgaccatccggaccatatatagacagctataccacacccgcgtggatgcatgcatggctcatcccttacttgtgatgtatagacaattcacactatatatatatatatatatatatatatatatatatatatatattgctttgatttttgatctctgatatatatatatcgccagcatattatcactggcaccacttatcctcgctcgatctctcagaaacaataataacatagagagtttatatagttcgaacattagtatcgttttttatttgcaatagattctaatgtaactctttagtactggttgtaacaaaaaaaaaccagtactacatactaagttttcaacaacaactgatactaaaattattttattagttttctacaaacaatatgatattaacagagagtacattaataattatgtgcatatacaaatatacaagttctttccaatggatatgtgcacatacaaaaatacaagtactttccaatagatatgagcagatacaaacatacaaacatccaaatatacatatagcttctttattgaatatataaattttaggagagtacatattatatatttttagtacaaggaaagtgtgaaaacacgtctgattcccttctctattttttcgtctcagttcctgcttcttcccaaacctattactgcaaccctacttcctcttgtgcggaggttaggtcaagggattccaaaccaactacgggtttaatgtgagggacgaagatgctcttacaaccaactagcccttgaagtaggaaatagaatgtattttcttatttcctgccataggactagttcatttttcaagatcttctccatttcactatgaccaatgcgaggatggtttctatgaaaaaaatatacaaaacagaaaaaatagagttcaaaacaaatggtgtaatagttatatatggacagtaaacaaaaaataaaaaacatggatgaatatcattttgcgaatgtgtaaaaaatactcgaggcacgtgacatttgtgttgtattgataattaacacactatgtgacatttactcaaaactttcattggtactcaacacgtccggtgctcacgaatgagaaaggcaggttgcgcttataggagacacccctagccaataacttgtaccaaatccttcacttttagtgttggttatttaaaagaatcggtgctaaactaattttggcgggaatttcaattgctctggcaggattggaaaatatctttctatatcggttctttataaaaaacggttctaaataaacacaatatttccaaccagtactaaagacttaaaatttagtatcgattctataaaagaaccgatactaaagatattggagatgttctcttcgaataatccatagagaaaaaacactgtggaagcttagtgcagattaaagtcaagcctaattagtgctggttcttaggttgaactagcagtgctagataacttaatattaagtattagttgcatacaacaacccatactgatatcatatcctctgcacggactatattttaaaacggtgctaaagctagtactaatattggaacaaataaaattggacggaaaccccattctccggtagggaataatgatttgtttttcttcttgcactttaatttgtttagtttatattctcgctagcttgacgacataaatacatgtgtgtagatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacagcggatatacctctctatcgatcaacagcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaccctttttttatcttccgttttctcaactctcttatcgatcacctgcataaaggacctttgtatcaagtaccaagagatcttgccatgagttgcactttacgcacatttttttttctttttttttttcaggaacgtactactcttcctatatatcaatatatgtaaacaagattaacatgcatgtttctaacctttctcaaagacaaaagacactctggtgcacgaaatggatggaagaaaccagatcattaatatatgcctcacaacctcttcatgaatttaatttgatgtggaaagaataaaaacgacggttccggttgttaacccaatattcaatgatatcctgaacaaaactagctatagatctcaatcatagcatcaggcatcagcgcttccaaagttctcacctgactttttttttactcaatctccagattatatttccttcctacaaagagtcggagagaacatagccatgagttaaatcactgatgttgtaaatacagaccagtagtcaaaagcattgactatacactaaaactattgttcaaggtcactatttcacaaaaaaattcattgcctatttgatagtttgattgagaggtagcaatattgccaaatttatttttcacgtacctagacaaaagtcggtagcaatattgccaattttattgctccatcgtcatatgcatcccgaagtctattattgctgtaatgacaagatacagatcttttatattgtgatatacttacttaagttttatattgaagataaaagggagaaagcagcttgcctccctttctttttcttcaccactatatatattggattgtttcttcaccactatatatacaagaaaatattaatatctgcagtacatatttagtgtcattaaatatgtcttttgaaactattttcataataaacatatttgaagatacatgtattgcaaatattttttacgaatctaatcaaatatgagaaattttgactgacatgtatgaccatactatcaattattttaggacagaggcatggagtgcgcatttgtatggtcgaaatcgatcaattgtaaccatatatgcatgtacgtttggtacgcccactgatgtatctacctggttaattaattagatgacctagcttgtcgtctgattgttatgattaaagaaccaaaaagtctactcagctcaaaacccaaatatatatgtgtcaaacaactcccatgcacatgtccagctgtgtctaaatctatcccgaaggattgtccatgccaaagtttgatgaaatagataataagttgtctcattttatgtggttcgtttttgcagatttgctgcttactctttcgtatacttggattttatagggaactaatatatacatatgattataattaatgcactttattccgtgccacatgtagatgaataacgcaatcacatggcttaagatctaatattctaccccaaaacaaatcgagctaccaaggcgatatctgatgttcatcaggcatgcatgtaggcccattcagcatatcaagcaaagtacagattcttatccaaaccatgcatatacatatgaccaaagtactaattaattagttgcctgcagttattagctgtccaaaatttgctttgatcatcatgcaataatatacacatgcagaaactaaaatgaataacatatataaatccatgcatgcacatgcagcatactaattattgctattaattaattgcagatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacgcctgaatcttatgtatatatgtatgtatagttagtaaatgtagtacccctctattcttttttatttgtcgccgattagtttaaaaataaactagcggacgacaaatattcgagaacagaggtagtatatgttatgtctcaattgccaactgcaatagcaaaacttttggggatactgtttgaattgctattttgtatgatagtatatattatgacgtcactaccggaattcgggcttttggcgagtgctaggagctttaccga

SEQ ID NO: 2 - DNA, 720 bp, Zea mays
atgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaa

SEQ ID NO: 3 - protein, 240 aa, Zea mays
MSSGPAACGVCGAAACCPHLLHTGDGNDDDLISRAFFSVFPVVGHHRRHESTSSPAMQQPSGCLHEFQFFGHQDDHHHQETIAWLLDHPPPPAPELGGDDGPSPAGDENDDQPAFHPFGTPQYHHPGKGNGNGLTFELDATLGLGTARQTTETAEASATIMSFSGSTFTDAASKEPALIDDGNELQMPVDQSSTEREVKLMRYKEKRMRRCFEKQIRYASRKAYAQVRPRVKGRFAKVTE

SEQ ID NO: 4 - DNA, 5009 bp, Zea mays
ctacacgcaaaagctatgcggattctgtttctgtgtaaacaaccaacactctctttattccaatggacaatgctccatctctcttgtgccccccgcaggacagaattggctaggcggcgattccacgtgccatcacatttccaattctgaagttagcctatcccaccgttgcttcctacacgtgtcgaccatccggaccatatatagacagctacaccagacccgcgcgtggatgcatgcatgggtcggtcatcccttgtgatgtatagaaaattcacgctaatataatatatatataacttgctttgatttttgatctctgatatatatcgtcgccagcatattatcactggcaccacttatcctcgctccatctctcagaaacaataataatatagagagtttatatagttcgaacattagtatcgtttttttatttgcaatagattctaatgtaactctttagtactggttgtaacaaaaaaaaacagtactaaatactaagttttcaacaacaactgatactaaaattattttattagttttctacaaacaatatgatatcgacaaagagtgcattaataattatgtgcgtatacaaatatacaagttctttccaatggataagtgccaatacaaaaatacaagttctttccaatagatatgggcggatacaaacatccaaatatacatatagcttgtttattaaatatataaatcttaggagagtattatatatttttagtacaaagaaagtgcgaaaacacgtctggttcccttctttattttttcgtctcagttcctttctttattttttcgtctcagttccttcttccctaacctactacgtacttcaaccctacttcctcttgtgcggaggttttaggtcaagggattccaaaccaacgagtttaatgtgagggacgaagatgctcttacaaccaactagcctttgaagcaggaaatagaatgtattttcttatttcctgctatagggctagttcatttttcaagatcttctccatttcactatgaccaatgcgaggatggtttctatgaaaaaaatatataaaacagaaaaaatcgagttcaaaacaaatgatgtactaattatggacgataaaaaaaacatgggtgaatatcattttgcgactgtgtaaagaatactcaaggcacatgacatttgtgttgtaccgatccttaacacactatgtgacatttactcaaaactttcattgtactcaacacgtccagtgctcacgaatgagaaaggcaggctgcgcttataggagacaccctagccaataacttgtactaaatccttcacttttagtgttggttatttaaaagaagcagtgctaaactaattttggcaggaattccaattgctctggcaggattggaaaatatctttctatatcggttcttataaaaaatggtactaaaggcatctctatttttaatttgagtacttggtcttgaggttctaaataaacgcaatatttccaaccagtactaaagacttaaaatttagtatcgattctataaaagaaccgatactaaagatattggagatgttctcttcgaataatccatagagaaaaaacactgtggaagcttagtgcagattaaagtcaagcctaattagtgctggttcttaggttgaactagccgtgctagataacttaatattaagtattagttgcatacaacaacccatactaatatcctctgcacgaactatatttaaaaacggtgctaaagctagtactaatattggaacaaatataattggacggaaaccccattctccggtagggaataatgatttgtttttcttcttgcactttaatttgtttagtttatattctcgctagcttgacgacataaatacatgtgtgtagatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacaacagcggatatacctctcgatcaacagcggccatgtcgtcggggccagcagcatgcggtgtgtgcggcgcggccgcctgctgcccgcacctcttgcacaccggtgacggcaacgacgacgacctcatcagccgagccttcttctccgtcttccctgtcgtcggtcatcaccgtcgtcatgagtccaccagcagccccgccatgcagcagccatcggggtgcctgcacgagttccagttctttggccaccaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaacctttttttttcttccgttttcgttctcaactctcttatcgatcacctgcataaaggaccttagtatcaagtaccaagagatcttgccatgagttgcactttacgcacatattttttttcttttttttttcaggaacgtactactcttcctatatatgcatgtaaacaagattaacatgcatgtttttctaacctttctcaaagacaaaagacactctggtgcacgaaatggatggaagaaaccagatcattaatatatgcctcacaaccttcatgaatttaatttgatgtggaaagaataaaaacgacggttccggttgttaacccaatattcaatgatatcctgaacaaaactagctatagatctcaatcatagcatcaggcatcagcgcttccaaagttctcacctgactttttttttactcaatctccagattatatttccttcctacaaagagttggagagaacatagccatgagttaaatcactgatgttgtaaatacagaccagtagtcaaaagcattgactatacactaaaattattgttcaaggtcactatttcacacaaaatttcattgcctatttgatagtttgattgagaggtagcaatattgccaaatttatttttcacgtacctagacaaaagtcggtagcaatattgccaattttattgctccatcttcatatccagcccgaagtctattattgctgtaatgacaagatacatatcttttatattgtgatatacttacttaagttatatattgacgataaaagggagaaagcagcttgcctccctttctttttcttcaccactatatatattggattgtttcttcaccacaatatacaagaaaatattaatatctgcagtacatatttggtgtcattaaatatgtcgtttgaaactattttcataataagcatatttgaagatacagatattgcaaatattttttacgaatctaatcaaatatgagaaattttgactgacatgtatgaccatactatcaattattttaggacagaggcaggaagtgcgcatttgtatggtcgaaatcgatcaattgtaaccatatgcatgtacgttggtacgcccactgatgtatctacctggttaattaattagatgacctagcttgtcgtctgattgttatatatgattaaagaaccaaaaagtctactcagctcaaaacccaaatatatatgtctcaaacaacttccatgcacatgtccagctgtgtctaaatctatcccgaaggattgtccatgccaaagtttgatgaaataggtaataagttgtctcattttatgtggttaatttgcagatttgctgcttactctttggtatacttggattttatagggaactaatatatacatatgattatgattaatgcactttatttcgtgccacatgtagatgaataactcaatcacatggcttaagatctaatattctaccccccaaaaacaaatggtgctaccaaggcaatatctgatgttcatcaggcatgcatgtagctaggcccattcagcatatcaagcaaagtacagattcttatccaaacaatatatatgaccaaagtactaattaattagttgcctgcagttagctgtccaaaatttgctttgatcatcatgcaataatatacacatgcagaaactaaaatgcaataacatatataaatacatgcatgcatgaacatgcatactaattgctattaaataattgcagatgtcattctgtgggagcacattcactgacgctgcaagcaaggagccagcactgatcgacgacggcaatgagctgcaaatgccggtagatcagtcgtcgtcggagagggaggttaagttgatgaggtacaaggagaagagaatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacggctgaatcttatgtatatatgtatttatagttagtaaatgtattactccctctattcttttttatttgtcaccgattagtttaaaaataaactagcggacgacaaatattcgagaacagagatagtatatgttatgtctcaattgccaactgcaatagcaaaacttttggggatact

SEQ ID NO: 5 - DNA, 724 bp, Zea mays
atgtcgtcggggccagcagcatgcggtgtgtgcggcgcggccgcctgctgcccgcacctcttgcacaccggtgacggcaacgacgacgacctcatcagccgagccttcttctccgtcttccctgtcgtcggtcatcaccgtcgtcatgagtccaccagcagccccgccatgcagcagccatcggggtgcctgcacgagttccagttctttggccaccaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcatgtcattctgtgggagcacattcactgacgctgcaagcaaggagccagcactgatcgacgacggcaatgagctgcaaatgccggtagatcagtcgtcgtcggagagggaggttaagttgatgaggtacaaggagaagagaatgaggaggtgctttgagaagcagataagatatgcatccaggaaagcctatgcgcaggtgagacccagggtgaaaggccgctttgccaaggtaaccgaatgac

SEQ ID NO: 6 - protein, 240 aa, Zea mays
MSSGPAACGVCGAAACCPHLLHTGDGNDDDLISRAFFSVFPVVGHHRRHESTSSPAMQQPSGCLHEFQFFGHQDDHHHQETIAWLLDHPPPPAPELGGDDGPSPAGDENDDQPAFHPFGTPQYHHPGKGNGNGLTFELDATLGLGTARQTTETAEASATIMSFCGSTFTDAASKEPALIDDGNELQMPVDQSSSEREVKLMRYKEKRMRRCFEKQIRYASRKAYAQVRPRVKGRFAKVTE

SEQ ID NO: 7 - DNA, 4813 bp, Zea mays
ctctggtgccccccgcaggacagaattggctaggcggcgattccacgtgccatcacatttccaattctgaagttagcctatcccaccgttgcttcctacacgtgtcgaccatccggaccatatatagacagctataccacacccgcgtggatgcatgcatggctcatcccttacttgtgatgtatagacaattcacactaataatataatatataacttgctttgatttttgatctctgatatatatcgccagcatattatcactgccaccacttatcctcgctccatctctcagaaacaataattcaggagccatgcatgtagtcatatcctccctaggtcgttctgttcattcctgcattaaaaaaatgtcaattgtaatgggtctaaaggtgcaagtaagtttgatattataatatggggagtttatatagttcgaacattagtatcgtcgttttttatttgcaatagattctaatgtaactcgttaattagtaccggttgtaacaaaaaaaaaaaccagtactaaatactaatttttcaacaacaactgatactaaaattattttattatttttctacaaacaatatgatatcaacagagagtacattaataattatgtgcctatacaaatatacaagttctttccaattgataagtgcacatacaaaaatacaagttctttccaatagatattagcaaatacaaacatacaaattaatatccaaatatacatatagcttctttattaaatatataaatcttaggaaactattatatatttttagtacaaaaaaagtgcgaaaacccgtcgggttcccttctctattttttcgtctcggttccttcttccctaatctactactgcaaccctacttcctcttgtggggaggttttaggtcaaggaattccaaaccaacgggtttaatgtgagggacgaaaatgctcttacaaccaactagcctttgaagcaggaaatagaatgtattttcttatttcctgctataggcctagttcatttttcaagatcttctccatttcactatgaccaatgcgaggatggtttctatgaaaaaaaatatataaaacagaaaaaatcgagttcaaaacaaatgatgtactaattatggacaataaacaaaaaataaaaacatgggtgaatatcattttgcgactgtgtaaagaatactcaaggcacgtgacatttgtgttgtaccgatacttaacacactatgtgacatttactcaaaactttcattggtactcaacacgtccagtgctcacgaatgagaaaggcaggctgcgcttataggagacaccctagccaataacttgtactaaatccttcacttttagtgttggttatttaaaagaatcggtgctaaactaattttggcgggaatttcaattgctctggcaggattggaaaatatctttctatatcggttcttataaaaaacggtactaaaggcatctctattttttgagtacttggtctcgaggttctaaataaacgcaatatttccaaccagtactaaagacttaaaatttagtatcgattctataaaagaaccgatactaaagatattggagatgttctcttcgaataatccatagagaaaaaacactgtggaagcttagtgcagattaaagtcaagcctaattagtgctggttcttaggttgaactagcagtgctagataacttaatattaagtattagttgcatacaacaacccatactaatatcatatcctctgcacggactatattttaaaacggtgctaaagctagtactaatattggaacaaataaaattggacggaaaccccattctccggtagggaataatgatttgtttttcttcttgcactttaatttgtttagtttatattctcgctagcttgacgacataaatacatgtgtaaatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacaacagcggatatacctctcgatcaacagcggccatgtcgtcggggccagcagcatgcggtgtgtgcggcgcggccgcctgctgcccgcacctcttgcacaccggtgacggcaacgacgacgacctcatcagccgagccttcttctccgtcttccctgtcgtcggtcatcaccgtcgtcatgagtccaccagcagccccgccatgcagcagccatcggggtgcctgcacgagttccagttctttggccaccaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaacctttttttttcttccgttttcgttctcaactctcttatcgatcacctgcataaaggaccttagtatcaagtaccaagagatcttgccatgagttgcactttacgcacatattttttttcttttttttttcaggaacgtactactcttcctatatatgcatgtaaacaagattaacatgcatgtttctaacctttctcaaagacaaaagacactctggtgcacgaaatggatggaagaaaccagatcattaatatatgcctcacaaccttcatgaatttaatttgatgtggaaagaataaaaacgaaggttccggttgttaacccaatattcaatgatatcctgaacaaaactagctatagatctcaatcatagcatcaggcatcagcgcttccaaagttctcacctgactttttttttactcaatctccagattatatttccttcctacaaagagttggagagaacatagccactgatgttgtaaatacagaccagtagtcaaaagcattgactatacactaaaactattgttcaaggtcactatttcacacaaaatttcattgcctatttgatagtttgattgagaggtagcaatattgccaaatttatttttcacgtacctagacaaaagtcggtagcaatattgccaattttattgctccatcttcatatccagcccgaagtctattattgctgtaatgacaagatacatatcttttatattgtgatatacttacttaagttatatattgacgataaaagggagaaagcagcttgcctccctttctttttcttcaccactatatatattggattgtttcttcaccacaatatacaagaaaatattaatatctgcagtacatatttggtgtcattaaatatgtcgtttgaaactattttcataataagcatatttgaagatacagatattgcaaatattttttacgaatctaatcaaatatgagaaattttgactgacatgtatgaccatactatcaattattttaggacagaggcaggaagtgcgcatttgtatggtcgaaatcgatcaattgtaaccatatgcatgtacgttggtacgcccactgatgtatctacctggttaattaattagatgacctagcttgtcgtctgattgttatatatgattaaagaaccaaaaagtctactcagctcaaaacccaaatatatatgtctcaaacaactcccatgcacatgtccagctgtgtctaaatctatcccgaaggattgtccatgccaaagtttgatgaaataggtaataagttgtctcattttatgtggttaatttgcagatttgctgcttactctttggtatacttggattttatagggaactaatatatacatatgattatgattaatgcactttatttcgtgccacatgtagatgaataactcaatcacatggcttaagatctaatattctaccccccaaaaacaaatggtgctaccaaggcaatatctgatgttcatcaggcatgcatgtagctaggcccattcagcatatcaagcaaagtacagattcttatccaaacaatatatatgaccaaagtactaattaattagttgcctgcagttagctgtccaaaatttgctttgatcatcatgcaataatatacacatgcagaaactaaaatgcaataacatatataaatacatgcatgcatgaacatgcatactaattgctattaaataattgcagatgtcattctgtgggagcacattcactgacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccggtttgccaaggtaaccgaatgacgaagcctgcta

SEQ ID NO: 8 - DNA, 720 bp, Zea mays
atgtcgtcggggccagcagcatgcggtgtgtgcggcgcggccgcctgctgcccgcacctcttgcacaccggtgacggcaacgacgacgacctcatcagccgagccttcttctccgtcttccctgtcgtcggtcatcaccgtcgtcatgagtccaccagcagccccgccatgcagcagccatcggggtgcctgcacgagttccagttctttggccaccaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcatgtcattctgtgggagcacattcactgacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccggtttgccaaggtaaccgaa

SEQ ID NO: 9 - protein, 240 aa, Zea mays
MSSGPAACGVCGAAACCPHLLHTGDGNDDDLISRAFFSVFPVVGHHRRHESTSSPAMQQPSGCLHEFQFFGHQDDHHHQETIAWLLDHPPPPAPELGGDDGPSPAGDENDDQPAFHPFGTPQYHHPGKGNGNGLTFELDATLGLGTARQTTETAEASATIMSFCGSTFTDAASKEPALIDDGNELQMPVDQSSTEREVKLMRYKEKRMRRCFEKQIRYASRKAYAQVRPRVKGRFAKVTE

SEQ ID NO: 10 - DNA, 4460 bp, Zea mays
gatacaacatacaaacatccaaatatacatatagcttctttattgaatatataaattttaggagagtacatattatatatttttagtacaaggaaagtgtgaaaacacgtctgattcccttctctattttttcgtctcagttcctgcttcttcccaaacctattactgcaaccctacttcctcttgtgcggaggttaggtcaagggattccaaaccaactacgggtttaatgtgagggacgaagatgctcttacaaccaactagcccttgaagtaggaaatagaatgtattttcttatttcctgccataggactagttcatttttcaagatcttctccatttcactatgaccaatgcgaggatggtttctatgaaaaaaatatacaaaacagaaaaaatagagttcaaaacaaatggtgtaatagttatatatggacagtaaacaaaaaataaaaaacatggatgaatatcattttgcgaatgtgtaaaaaatactcgaggcacgtgacatttgcgttgtattgataattaacacactatgtgacatttactcaaaactttcattggtactcaacacgtccggtgctcacgaatgagaaaggcaggctgcgcttataggagacacccgtagccaataacttgtaccaaatcattcacttttagtgttggttgtttaaaagaagcggtgctaaactaattttggcgggaattccgatccaattgctctggcaggattggaaaatatctttctatatcggttcttataaaaacggtactaaaggcatctctgtttttttgagtacttgttcttgaggtactaaataaacgcaatatttccaactagtactaaagagttaaaatttagtatcgattctataaaagaaccgatactaaagatatttgggatgttctattcgaattaaagccggctatttggactgctgcaactgtaatccatagagagaaaaaacactgtggaagcttggtacagattaaagtcaagcataattagtgctggttcttaggttgaactagcagtgctagataacttaataataagtatatattggttgtagacaacaaccggtactaatatcctctggacgaactatattttataacggtgctgaagttagtactaatattggaacaaatataatgagacggaaaccccattctccggtagggaataatgatttgtttttcttcttgcactttaatttgtttagtttatattctcgctagcttgacgacataaatacatgtgtaaatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacagcggatatacctctctatcgatcaacagcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccctagctggtgatgagaacgacgaccagcctgcgtttcacccgtttgggacaccacagtaccaccaccccagaaaagggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaccctttttttatcttccgttttctcaactctcttatcgatcacctgcataaaggacctttgtatcaagtaccaagagatcttgccatgagttgcactttacgcacatttttttttctttttttttcaggaacgtactactcttcctatatatcaatatatgtaaacaagattaacatgcatgtttctaacctttctcaaagacaaaagacactctggtgcacgaaatggatggaagaaaccagatcattaatatatgcctcacaacctcttcatgaatttaatttgatgtggaaagaataaaaacgacggttccggttgttaacccaatattcaatgatatcctgaacaaaactagctatagatctcaatcatagcatcaggcatcagcgcttccaaagttctcacctgactttttttttactcaatctccagattatatttccttcctacaaagagtcggagagaacatagccatgagttaaatcactgatgttgtaaatacagaccagtagtcaaaagcattgactatacactaaaactattgttcaaggtcactatttcacaaaaaaattcattgcctatttgatagtttgattgagaggtagcaatattgccaaatttatttttcacgtacctagacaaaagtcggtagcaatattgccaattttattgctccatcgtcatatgcatcccgaagtctattattgctgtaatgacaagatacagatcttttatattgtgatatacttacttaagttttatattgaagataaaagggagaaagcagcttgcctccctttctttttcttcaccactatatatattggattgtttcttcaccactatatatacaagaaaatattaatatctgcagtacatatttagtgtcattaaatatgtcttttgaaactattttcataataaacatatttgaagatacatgtattgcaaatattttttacgaatctaatcaaatatgagaaattttgactgacatgtatgaccatactatcaattattttaggacagaggcatggagtgcgcatttgtatggtcgaaatcgatcaattgtaaccatatatgcatgtacgtttggtacgcccactgatgtatctacctggttaattaattagatgacctagcttgtcgtctgattgttatgattaaagaaccaaaaagtctactcagctcaaaacccaaatatatatgtgtcaaacaactcccatgcacatgtccagctgtgtctaaatctatcccgaaggattgtccatgccaaagtttgatgaaatagataataagttgtctcattttatgtggttcgtttttgcagatttgctgcttactctttcgtatacttggattttatagggaactaatatatacatatgattataattaatgcactttattccgtgccacatgtagatgaataacgcaatcacatggcttaagatctaatattctaccccaaaacaaatcgagctaccaaggcgatatctgatgttcatcaggcatgcatgtaggcccattcagcatatcaagcaaagtacagattcttatccaaaccatgcatatacatatgaccaaagtactaattaattagttgcctgcagttattagctgtccaaaatttgctttgatcatcatgcaataatatacacatgcagaaactaaaatgaataacatatataaatccatgcatgcacatgcagcatactaattattgctattaattaattgcagatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacgcctgaatcttatgtatatatgtatgtatagttagtaaatgtagtacccctctattcttttttatttgtcgccgattagtttaaaaataaactagcggacgacaaatattcgagaacagaggtagtatatgttatgtctcaattgccaactgcaatagcaaaacttttggggatactgtttgaattgctattttgtatgatagtatatattatgacgtcactaccggaattcgggcttttggcgagtgctaggagctttaccga

SEQ ID NO: 11 - DNA, 731 bp, Zea mays
atgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccctagctggtgatgagaacgacgaccagcctgcgtttcacccgtttgggacaccacagtaccaccaccccagaaaagggaacgggctcacctttgagctggacgccacgctgggcctcggcaccgcgcggcaaaccactgagacagcagaagcaagcgccaccatcatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagc

SEQ ID NO: 12 - protein, 238 aa, Zea mays
MSSGPAACGVCGAAACCPHLLHTGDGNDDDLISRAFFSVFPVVGHHRRHESTSSPAMQQPSGCLHEFQFFGHQDDHHHQETIAWLLDHPPPPAPELGGDDGPSLAGDENDDQPAFHPFGTPQYHHPRKGNGLTFELDATLGLGTARQTTETAEASATIMSFSGSTFTDAASKEPALIDDGNELQMPVDQSSTEREVKLMRYKEKRMRRCFEKQIRYASRKAYAQVRPRVKGRFAKVTE

SEQ ID NO: 13 - DNA, 4398 bp, Zea mays
gatacaacatacaaacatccaaatatacatatagcttctttattgaatatataaattttaggagagtacatattatatatttttagtacaaggaaagtgtgaaaacacgtctgattcccttctctattttttcgtctcagttcctgcttcttcccaaacctattactgcaaccctacttcctcttgtgcggaggttaggtcaagggattccaaaccaactacgggtttaatgtgagggacgaagatgctcttacaaccaactagcccttgaagtaggaaatagaatgtattttcttatttcctgccataggactagttcatttttcaagatcttctccatttcactatgaccaatgcgaggatggtttctatgaaaaaaatatacaaaacagaaaaaatagagttcaaaacaaatggtgtaatagttatatatggacagtaaacaaaaaataaaaaacatggatgaatatcattttgcgaatgtgtaaaaaatactcgaggcacgtgacatttgcgttgtattgataattaacacactatgtgacatttactcaaaactttcattggtactcaacacgtccggtgctcacgaatgagaaaggcaggctgcgcttataggagacacccgtagccaataacttgtaccaaatcattcacttttagtgttggttgtttaaaagaagcggtgctaaactaattttggcgggaattccgatccaattgctctggcaggattggaaaatatctttctatatcggttcttataaaaacggtactaaaggcatctctgtttttttgagtacttgttcttgaggtactaaataaacgcaatatttccaactagtactaaagagttaaaatttagtatcgattctataaaagaaccgatactaaagatatttgggatgttctattcgaattaaagccggctatttggactgctgcaactgtaatccatagagagaaaaaacactgtggaagcttggtacagattaaagtcaagcataattagtgctggttcttaggttgaactagcagtgctagataacttaataataagtatatattggttgtagacaacaaccggtactaatatcctctggacgaactatattttataacggtgctgaagctagtactaatattggaacaaatataattggacggaaaccccattctccggtagggaatatatgatttgtttttcttcttgcactttaatttgtttagtttatattctcgctagcttgacgacataaatacatgtgtaaatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacagcggatatacctctctatcgatcaacagcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaccctttttttatcttccgttttctcaactctcttatcgatcacctgcataaaggacctttgtatcaagtaccaagagatcttgccatgagttgcactttacgcacattttttttttctttttttttttcaggaacgtactactcttcctatatatcaatatatgtaaacaagattaacatgcatgtttctaacctttctcaaagacaaaagacactctggtgcacgaaatggatggaagaaaccagatcattaatatatgcctcacaacctcttcatgaatttaatttgatgtggaaagaataaaaacgacggttccggttgttaacccaatattcaatgatatcctgaacaaaactagctatagatctcaatcatagcatcaggcatcagcgcttccaaagttctcacctgactttttttttactcaatctccagattatatttccttcctacaaagagtcggagagaacatagccatgagttaaatcactgatgttgtaaatacagaccagtagtcaaaagcattgactatacactaaaactattgttcaaggtcactatttcacaaaaaaattcattgcctatttgatagtttgattgagaggtagcaatattgccaaatttatttttcacgtacctagacaaaagtcggtagcaatattgccaattttattgctccatcgtcatatgcatcccgaagtctattattgctgtaatgacaagatacagatcttttatattgtgatatacttacttaagttttatattgaagataaaagggagaaagcagcttgcctccctttctttttcttcaccactatatatattggattgtttcttcaccactatatatacaagaaaatattaatatctgcagtacatatttagtgtcattaaatatgtcttttgaaactattttcataataaacatatttgaagatacatgtattgcaaatattttttacgaatctaatcaaatatgagaaattttgactgacatgtatgaccatactatcaattattttaggacagaggcatggagtgcgcatttgtatggtcgaaatcgatcaattgtaaccatatatgcatgtacgtttggtacgcccactgatgtatctacctggttaattaattagatgacctagcttgtcgtctgattgttatgattaaagaaccaaaaagtctactcagctcaaaacccaaatatatatgtgtcaaacaactcccatgcacatgtccagctgtgtctaaatctatcccgaaggattgtccatgccaaagtttgatgaaatagataataagttgtctcattttatgtggttcgtttttgcagatttgctgcttactctttcgtatacttggattttatagggaactaatatatacatatgattataattaatgcactttattccgtgccacatgtagatgaataacgcaatcacatggcttaagatctaatattctaccccaaaacaaatcgagctaccaaggcgatatctgatgttcatcaggcatgcatgtaggcccattcagcatatcaagcaaagtacagattcttatccaaaccatgcatatacatatgaccaaagtactaattaattagttgcctgcagttattagctgtccaaaatttgctttgatcatcatgcaataatatacacatgcagaaactaaaatgaataacatatataaatccatgcatgcacatgcagcatactaattattgctattaattaattgcagatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacgcctgaatcttatgtatatatgtatgtatagttagtaaatgtagtacccctctattcttttttatttgtcgccgattagtttaaaaataaactagcggacgacaaatattcgagaacagaggtagtatatgttatgcatgtctcaattgccaactgcaatagcaaaacttttggggatactgtttgaattgc

SEQ ID NO: 14 - DNA, 765 bp, Zea mays
atgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacgcc

SEQ ID NO: 15 - protein, 240 aa, Zea mays; identical to SEQ ID NO: 3

SEQ ID NO: 16 - DNA, 4728 bp, Sorghum bicolor
ctctctctattttcaccatttcaaaaagcaaaaagctaaagccaaaaataaagtcctaggtgttctgaaaactgtactgccaaaaagtagctgcttttgaaaaagcagcttacggttttcactcatttgttggttagcttttgtctttttagaagtaaaaatgtgtaaattttaggagcataaattaggagttattggagagagattatttttttctcatctggctaagaactaaggattgagaagggaaatgggaaattcttggagatactcttaggaggagctataataattttataaatagtggttatgataactcaaagtgacaatagaataagctatttatagttgataacaaactatcatatgttttttgttaatttgacgcatgctcgatcgatacaagagcttgtgtgttagcacgcgtgccttgcatcgatctgctgttatggtagtgtactaattctctgctgtcgtcgctgcccctagaggtggtgcagtgcgtcgtggagcgaggaagaatgcccatcacacatgaaagcctcatatccttgcagtcaaacgtggatctcctgcgtccaagccttcctccatgagtggatgaaatggacccgtacttctgttttcaccttttgggaacattcaagaaagcaacccttgtcgcagcatgattcacatgattattcttcgcgcaccaacaaaattcaaccagaggaaatttaatttgtagattgtaggtaacaaaatgagaaaacgaaatatataattaaaggacatatatggaccccaacttatttggagttcctacttgaaatcagattgtacaaaactcaaaggtatctggtgttgtgggtgcgatgtgcactcatattccttttgttgtatgattttcgttccatactaagggcctgtttagatcccaaagctttacattccaaaactgttggttgtaaattttacattccaaataaatgtttagatgcttttcaaatttttttatctacaacacataagacacgggctcctaaataagtttatacagttttgggctccaaggtcacaggtccctgtttagatttattcttccaaaaagtgctcatttctccaaaagttttggttgtttggccgcatctaaacagggcctagatcatttgagacagggtgcgcatcacctaccagttgatgacaaatgttccattatttttgcattttttagcagatctttagatccattcttcctaaaagtgctcatttctccaaaagttttaactgtttggctgcatctaaacagggcctagatcatttgagacagggtgcgcatcacctaccagttgatgacaaatgttccattatttttgcattttgtagcagattatatgaaaagcactgaaaaaggaacttgtgtagccactttgtgatgcaaaagttagttaaaagaagactacacactagtaatttactgacgatcaaccacagtacagcacaaacaaccaaatcgatccacagaagaaataaaaaaagagaatcacccaagatgcatcatatgcattgattggcaggggatggccaatggggaatcgccaaatgatccagcctctggagggacaagaaaggtggctccggatcctccacgtaagaatccctcccatctcatatctcttgccccgctctcattggccgcatccgcatcacggcgcgcgcgtacttattcctccccccgccctagcctcctcctctccctctcttctcctccacacaccaccacaccaagcctgcccgccccgccgagcgaagcgagagagctagcggccggccggccgggcaagcaaacaagcaagtggagggagagacagagagagagagagacagagctcatcacaaaccacacaggaaggtgtgtggtggaaggctggaggaggaagagagaggggccggccggggtcgtcaagagagtgagttcatatattcatacgtgcggcggcgacgtcgtcgtacgcaggatgtcggcgtcggcgtcgagcgccgcgtgccgcgtgtgcggtggcggcgtaggggagtgcgcgtgccaccacggccatggtgggatcggcggtggcgcgcggtgcggggtcgctgtcgcggacctcaaccgcgggtttcccgggatgtggcatcaccagcaccagccggaggaggaacccggcggcgtcgtcgtcggcagtggcgcggcggcggcggcggccgggctgcatgagttccagttcttcggccacgacgaggaccacgacagcgtgacgtggctgttcaacgaccccgcgccacacctgcaccgtggcccggcgccggctgccacggtcgggaacggggtggccgccgaggctgctgagcagccgagagcgccgccgccgttgttcgacgggtacgcgcacgcgcacgcgccgtacgggcagctgccgggtcacgggctcacgttcgacgtgccgctgggccggggcggcgaggtgcaggcctccgcggcggtgctggaggcagggctggggctaggcggcggcggcggtggcggcggcagcaatccggcgacgacgtccagcggcgccacaatcgtgagtggggttcggttccatctctctcctgaatcatgcatgcatggtcccccgtcgacaagtccaatttcattgtgtgctggcgatatgcatgcgattctcgtgtcacgtttgtgcacactgaaccgctgatatgtctttgcaggcttgcagaactgctcggccatttcagtttcgaaatacatgcatcttggtgcaatctatcgctcgcgcttcttgttaaacctctcggttcgtcgtgcccagttaattgctttgcttgatgcttgagagagaaggaagctacacctgatttcgctagcttagtttttgcattggtacatatctttatctttatctagctgtttggccaattaaaagtgattgaaaacgtcaaaaagagccgctgtcaatttgtggtccaacgctccaactagtaaggactgaggagctaactagtccaaaacagtgcaccattatgactaataagttacagtccgtcttgttttcttcttcgtctctacctttccaataggataaaatcacattatcaaagcaatactttaatttgttgtatacatatatccatgaaattcctgagaatcttaagagctgcatcaacgcttattagatcggacagctctatctatgctaagaagaggtacagaaattgaaaaccacatgcatgcacactatttttttttgaagacactggtttgtagtatatgcagagatagatgggaacacaatcacggcccagttacaaacgcaaacgcttatatacacgagcgcacaatcacctctatgaatgcacgtatgcacgttctatctctataagcacctccgaagaaccaagttggatcgacaaatctcaagattggacaaagtcaccacacccgagtagacaagttccaccacatggaaccgaagcagctaatctatgatcagttcgcttagagttgatttatctgttgagtggctagtgagaacactaaaaggtagtatgataacataacaaaattttgattgggatgcacaaatgaattggaacaaattccctgcccaaatgcatggttttcctttttccttcacacgatcagtatcgctatctttttgacaacatgataaccacaactccacaagaccttgccaaacgcaattttctttcttttctttctttcttgctatcgctatctgtctatcaacaagctcacgacgggtcgtgaagataaccctggccaacacgcagctcaattctcatttcatgtggttctacgatattgatcccaacttctcattatttctgttgagtagtgttagattaattactaaatgctgacatagtgtatccgatttccgaacgtaaatccgaagcaatgttatgcatcgcacctagctcttgtccaatagaagttttttacaacatgtaaggttaattacatttaccgggccgacacgtatatatatatatatctaattaaccacaaggatgatcaactgatctagataaagtaatgctttgtgggaccatatttgtgcatatacttgatagattagtagatcgtatagtttttttttagaagccacgtatcgtccaccaaggacaggggtccatctgagagaattggaccctgggtcatcgtctatgaaattttgttccattatttatgatcaaattaggatggcgctgcttaattagatgcatctaggaatatcgtaagtgggtacctaaagatgttctgtgtgggtcagctagtctagttcgttttcttcccctacatgatggctacatgcattacatccctacatgcatcggtgccttaactattatgttcgacacgtcctttttcagatgtccttgtgcgggagcacgttcactgacgcggcgagcgccgtcccgggcgacgcggccaacggcagcgcgagcggcgccgacccggcggtggaccgggaggcgaaggtgatgcgatacaaggagaagcgcaagaggaggcgctacgagaagcagattcggtacgcctcccgcaaggcctacgcagagatgcggccgcgcgtcaagggccggttcgccaaggtgcccgacggtgacgctccggcgccaccggcgccggccgcagcagcaggctacaaaccaggccggctcgacctcggatggttccgttcatag

SEQ ID NO: 17 - DNA, 897 bp, Sorghum bicolor
atgtcggcgtcggcgtcgagcgccgcgtgccgcgtgtgcggtggcggcgtaggggagtgcgcgtgccaccacggccatggtgggatcggcggtggcgcgcggtgcggggtcgctgtcgcggacctcaaccgcgggtttcccgggatgtggcatcaccagcaccagccggaggaggaacccggcggcgtcgtcgtcggcagtggcgcggcggcggcggcggccgggctgcatgagttccagttcttcggccacgacgaggaccacgacagcgtgacgtggctgttcaacgaccccgcgccacacctgcaccgtggcccggcgccggctgccacggtcgggaacggggtggccgccgaggctgctgagcagccgagagcgccgccgccgttgttcgacgggtacgcgcacgcgcacgcgccgtacgggcagctgccgggtcacgggctcacgttcgacgtgccgctgggccggggcggcgaggtgcaggcctccgcggcggtgctggaggcagggctggggctaggcggcggcggcggtggcggcggcagcaatccggcgacgacgtccagcggcgccacaatcatgtccttgtgcgggagcacgttcactgacgcggcgagcgccgtcccgggcgacgcggccaacggcagcgcgagcggcgccgacccggcggtggaccgggaggcgaaggtgatgcgatacaaggagaagcgcaagaggaggcgctacgagaagcagattcggtacgcctcccgcaaggcctacgcagagatgcggccgcgcgtcaagggccggttcgccaaggtgcccgacggtgacgctccggcgccaccggcgccggccgcagcagcaggctacaaaccaggccggctcgacctcggatggttccgttcatag

SEQ ID NO: 18 - protein, 298 aa, Sorghum bicolor
MSASASSAACRVCGGGVGECACHHGHGGIGGGARCGVAVADLNRGFPGMWHHQHQPEEEPGGVVVGSGAAAAAAGLHEFQFFGHDEDHDSVTWLFNDPAPHLHRGPAPAATVGNGVAAEAAEQPRAPPPLFDGYAHAHAPYGQLPGHGLTFDVPLGRGGEVQASAAVLEAGLGLGGGGGGGGSNPATTSSGATIMSLCGSTFTDAASAVPGDAANGSASGADPAVDREAKVMRYKEKRKRRRYEKQIRYASRKAYAEMRPRVKGRFAKVPDGDAPAPPAPAAAAGYKPGRLDLGWFRS

SEQ ID NO: 19 - DNA, 4643 bp, Sorghum bicolor
atatattcagcttagctgaggatccaatggagcacagctagctttagttaaagcaaacaaaattcaaagcaaaatgggtagggccagcgatcgattcctacgctcttttctgtgttccctgtcgctgccattgccatcgtggctgcaatatgtgtgtgcctatctgtcgccaccagggctccgctacccaaaggtaggattccgtttctgtgtaaacaaaccaacgacgtctttattcactacccaaatgcaccatgcattgcattgcatcttcctgttcgagagttttttaaaagccaacacttatatatactttctttaggactaattggtagtaagcactttaacaagaaatgtactggcaatagctactactagtgtgctactctcggtgatcaaaaaatacttgtcatattttgctttgcacggtattaataattttatgtgtattttgacaactaattaactttttgtataatacaaccaaacttacaatctcttaggcactacgccagaacacgcaatcactactaggtcttaacataggtatataatatataacatgctacaccagaacacgtaatcactactgccaggtcacttaacacaggtatataataacatgctttattgacctggcagtgatagcgtattatcactgtagggacgaccatatataataaacctataatgataactagccccatcactaccgattgtcctaatcaattgcgatggtgatggaggtttcttagccgacattgattgtgtgtacaggttaaggacttcaacttagacgaataggttataccataagaaacccaaccaagtgaggtacacacagttcacatgtgtgttgagactacttatgcatattaacgtgagaatatccacactacaactaaaggatcggagctagaagtcagagctagcagaaaggtggcatccctaaggtcttgtttagttcgttaaaatttttagatttcgttactatagcattttcgtttttatttaataattattgtccaatcataaactacctaggctcgaaagattcgtctcgcaaattacatataaactgcgtaattagttatcttttttatctatatatctaatgcttcatgcatgtaccgtaagatttgatgtaacggagaattttgaaaaaaaaaatggaactaaacaagtcctaagttaagttatattatccacccaaatgctttggttttcttcgtgcactcttgtttcgtttctttatctgatccacacatccagaggcaataactccttgtagtacttatcggtgcaaagatgctctcacacctaaactgcaagctgcctagctagctagcttcgatagatagatagataggtagatgaaacagaaggagtcatagcccctactgaaattaaattaaacaaagaaatacaacaataagcagtagctaaagaaaaaataaacctaaatatgtattatgtctagatacataacgaaagctatgtgtgtgtatatttagaaaaaactagaacgttagtcgattttgaactaataatttatttaagcgttgctagctgaccggcacgttatctccctgctgccacggtataccagatcgaatcctgtccatatatatctattctgccgcgggcactggctaccagattttgcatgcgatgcacacatctgcttttctgctgccttcccctggcctttcctttttcagtgcttaactgccgcgcgctacccccatggaattataaccctagcagctatagctatcaagctttatttattccttccttccttcctcttctagcagctacactacaccctgaactgcctcgatcaatatatatataactcaattcaacaaagtcatcatctccctccccagttgccgatcgatcgatcgatccatccatcttttaatttgctccaaacctatccacacagcagacctgagatcgagctgtgcgcgcgatcgatcgatcgatccgttcacatgtcagggccagcatgcggtgtgtgcggtgcagccgcctgctgccggcacctcttccacaccggcgacgagaacgacgacttcaacagccggcgggccttgttctctgtcttccctgcggcggttcaccatcatgagcccagccccagcagcatgcagcagcagcctccggcggggtgcctgcacgagttccagttctttggccatcaggacaacgatgaccaccaagaaagcatcgcctggctcttcgaccacccgccgccacctgcgcatgatgtcgagtcgacgacgacgaccggtccccagctgagaaccagcagcctcatcaccgggcgtttgacccgtttgggacggagggaaacgggctcacctttgaggttgatgcccggctgggcctcggcagcgggggcgccgcccggcaaacagcagagacagcagcagcaagcgccaccatcgtaagtagtacagagatcgagagcatggaaagatataatgaatctcaggatcactcggctgactccaattttctgttcttgatttgtcttcagttgcttcacgttcttttctgttcttgattgatcacgattctttctttttcagtgcttgctttttattttagactagatccagagcgagctcacacaatatgtggttcctcctttttttttattttgaaagatcatatgtggttcttcttgcgatccttctctctccgcgtgtgtgtgtcacttctttttatttggtgccagcagaaaaaccagatttcggggtcttgctgactaagagaattttctatcatgcatggcgtatgagggcatatgcatgttttgcaactagaatctatgggttgagagaattgaacgattacttttttctgaataaatgggaaaaaaacaattacaagggtgttgtggaattttgttgttgatatataaccgacaaattcatcatcgatttgatgagagtaaactaaatactctcatacatagagtttgtgcgaggactacgtactgagatggatattaataaatccaaattaatcacctttttggagaggaagaaatagatatggaaaagggtaagattaattgttgtatatacttactaattaactgaattgatcatatattcttatcagcaaatctatatattgtgatatagttactaagaaattatatatagataaaaggaagaaagcttggccttatttttccattttcttcatcactatattggttcattttgctcttgaaaatttatgcttgtctcatgatgatatatatagttaaccgcaggcactccagatttgtatggtttgaaatcaactgtaacaatgtatatgcatgtcggtgcatccactgatgcatccacctatttggatgacctagcttgtgtgattgttatgataaagaaaagaacaaaatatagtcctctaaaaccaaatataacatgtcaaacaatttccatgcatatgccctactggtatctcaacctgtcctgaaggtttgtccatgccaaagtttgattgaataggtaataagttgtctcattttatgtggatcatttgcatatttgctgtttgctttttgctacatttggcttttacaaaagtaataggtgattaattaatgcactttcattgcatatacgcacaaacaacttaatcacttgtcttaagatctaatttgtaccccaaacaaatcgtgctaccaaggcaaggaaatcagcatatacagatatagtcaatatttttccttgattatttggttatttaattgtctagtatgttctaaaatttaagggttgtgatttatttaaactcttacctcttaagaggtaataggtgtaccatagcaaaactctccgatgttcatcagacatgtaggccaattcaacatccagcaaagtgcagattcatatatcgaaacaataaatggcgaaggcatgcaacgagttatttagttacctagctctcctaattttgctttgatcatgcaagacaaagaaactaatatgaatgcaataattcatgcttaaccacaacctctttctggcccatttcttttattctccactgatttaggccttgtttagttccgaaaaattttaggaaatcgacactgtagcactttcgtttgtatttgacaaatattatctaatcatggactaactaggcttaaaagattcgtctcgtcaatttcgaccaaactgtgcaattagtttttatttttgtttatatttaatatcccatgcatgtgtctaaagattcgatgtgacggggaatgtgaaaaattttgcaaatttttcagggaagtaaacaaggccttaaagttgctgctctggttcatgcagatgtcattctgtgggagcacattcacagacgccgcaagctcgaggctcaaggagccaaccctgactgacgacagtcagctgcaaatgccggtaggtcagtcaacggagagggaggctaagttgatgaggtacaaggagaagaggatgaggaggtgttatgagaagcagataagatatgcatccaggaaagcctatgcgcaggtgagaccccgggtgaaaggtcgctttgccaaggtaaccgaagcctgctccgccacagcagacaatgttggcaacgaccacctgctctga

SEQ ID NO: 20 - DNA, 738 bp, Sorghum bicolor
atgtcagggccagcatgcggtgtgtgcggtgcagccgcctgctgccggcacctcttccacaccggcgacgagaacgacgacttcaacagccggcgggccttgttctctgtcttccctgcggcggttcaccatcatgagcccagccccagcagcatgcagcagcagcctccggcggggtgcctgcacgagttccagttctttggccatcaggacaacgatgaccaccaagaaagcatcgcctggctcttcgaccacccgccgccacctgcgcatgatgtcgagtcgacgacgacgaccgctgagaaccagcagcctcatcaccgggcgtttgacccgtttgggacggagggaaacgggctcacctttgaggttgatgcccggctgggcctcggcagcgggggcgccgcccggcaaacagcagagacagcagcagcaagcgccaccatcatgtcattctgtgggagcacattcacagacgccgcaagctcgaggctcaaggagccaaccctgactgacgacagtcagctgcaaatgccggtaggtcagtcaacggagagggaggctaagttgatgaggtacaaggagaagaggatgaggaggtgttatgagaagcagataagatatgcatccaggaaagcctatgcgcaggtgagaccccgggtgaaaggtcgctttgccaaggtaaccgaagcctgctccgccacagcagacaatgttggcaacgaccacctgctctga

SEQ ID NO: 21 - protein, 245 aa, Sorghum bicolor
MSGPACGVCGAAACCRHLFHTGDENDDFNSRRALFSVFPAAVHHHEPSPSSMQQQPPAGCLHEFQFFGHQDNDDHQESIAWLFDHPPPPAHDVESTTTTAENQQPHHRAFDPFGTEGNGLTFEVDARLGLGSGGAARQTAETAAASATIMSFCGSTFTDAASSRLKEPTLTDDSQLQMPVGQSTEREAKLMRYKEKRMRRCYEKQIRYASRKAYAQVRPRVKGRFAKVTEACSATADNVGNDHLL

SEQ ID NO: 22 - DNA, 44 bp, Artificial sequence
  primer
aattaaccctcactaaagggacggaaaccccattctccggtagg

SEQ ID NO: 23 - DNA, 47 bp, Artificial sequence
  primer
gtaatacgactcactatagggctgtccagcttgggaccaaattccaa

SEQ ID NO: 24 - DNA, 47 bp, Artificial sequence
  primer
aattaaccctcactaaagggcaagcgccaccatcgtaagtattgctc

SEQ ID NO: 25 - DNA, 49 bp, Artificial sequence
  primer
gtaatacgactcactatagggcacacagctggacatgtgcatgggagtt

SEQ ID NO: 26 - DNA, 49 bp, Artificial sequence
  primer
gtaatacgactcactatagggcgacagaggcatggagtgcgcatttgta

SEQ ID NO: 27 - DNA, 46 bp, Artificial sequence
  primer
aattaaccctcactaaagggccaaaagcccgaattccggtagtgac

SEQ ID NO: 28 - DNA, 26 bp, Artificial sequence
  primer
tatcgatcaacagcggccatgtcgtc

SEQ ID NO: 29 - DNA, 28 bp, Artificial sequence
  primer
tagctagcaggcttcgtcattcggttac

SEQ ID NO: 30 - DNA, 21 bp, Artificial sequence
  primer
cgccaccatcatgtcattctc

SEQ ID NO: 31 - DNA, 20 bp, Artificial sequence
  primer
tcagtgctggctccttgctt

SEQ ID NO: 32 - DNA, 18 bp, Artificial sequence
  probe
tgggagcacattcacgga

SEQ ID NO: 33 - DNA, 2996 bp, Zea mays
tctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacagcggatatacctctctatcgatcaacagcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagcctgcgtttcacccgtttgggacaccacagtaccaccaccccggaaaagggaacgggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaccctttttttatcttccgttttctcaactctcttatcgatcacctgcataaaggacctttgtatcaagtaccaagagatcttgccatgagttgcactttacgcacattttttttttctttttttttttcaggaacgtactactcttcctatatatcaatatatgtaaacaagattaacatgcatgtttctaacctttctcaaagacaaaagacactctggtgcacgaaatggatggaagaaaccagatcattaatatatgcctcacaacctcttcatgaatttaatttgatgtggaaagaataaaaacgacggttccggttgttaacccaatattcaatgatatcctgaacaaaactagctatagatctcaatcatagcatcaggcatcagcgcttccaaagttctcacctgactttttttttactcaatctccagattatatttccttcctacaaagagtcggagagaacatagccatgagttaaatcactgatgttgtaaatacagaccagtagtcaaaagcattgactatacactaaaactattgttcaaggtcactatttcacaaaaaaattcattgcctatttgatagtttgattgagaggtagcaatattgccaaatttatttttcacgtacctagacaaaagtcggtagcaatattgccaattttattgctccatcgtcatatgcatcccgaagtctattattgctgtaatgacaagatacagatcttttatattgtgatatacttacttaagttttatattgaagataaaagggagaaagcagcttgcctccctttctttttcttcaccactatatatattggattgtttcttcaccactatatatacaagaaaatattaatatctgcagtacatatttagtgtcattaaatatgtcttttgaaactattttcataataaacatatttgaagatacatgtattgcaaatattttttacgaatctaatcaaatatgagaaattttgactgacatgtatgaccatactatcaattattttaggacagaggcatggagtgcgcatttgtatggtcgaaatcgatcaattgtaaccatatatgcatgtacgtttggtacgcccactgatgtatctacctggttaattaattagatgacctagcttgtcgtctgattgttatgattaaagaaccaaaaagtctactcagctcaaaacccaaatatatatgtgtcaaacaactcccatgcacatgtccagctgtgtctaaatctatcccgaaggattgtccatgccaaagtttgatgaaatagataataagttgtctcattttatgtggttcgtttttgcagatttgctgcttactctttcgtatacttggattttatagggaactaatatatacatatgattataattaatgcactttattccgtgccacatgtagatgaataacgcaatcacatggcttaagatctaatattctaccccaaaacaaatcgagctaccaaggcgatatctgatgttcatcaggcatgcatgtaggcccattcagcatatcaagcaaagtacagattcttatccaaaccatgcatatacatatgaccaaagtactaattaattagttgcctgcagttattagctgtccaaaatttgctttgatcatcatgcaataatatacacatgcagaaactaaaatgaataacatatataaatccatgcatgcacatgcagcatactaattattgctattaattaattgcagatgtcattctctgggagcacattcacggacgctgcaagcaaggagccagcactgatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacgcctgaatcttatgtatatatgtatgtatagttagtaaatgtagtacccctctattcttttttatttgtcgccgattagtttaaaaataaactagcggacgacaaatattcgagaacagaggtagtatatgttatgcatgtctcaattgccaactgcaatagcaaaacttttggggatact

SEQ ID NO: 34 - DNA, 5073 bp, Zea mays spp parviglumis
gcggattctgtttctgtgtaaacaaccaacactctctttattccaatggacaatgctccatctctcttgtgccccccgcaggacagaattggctaggcggcgattccacgtgccatcacatttccaattctgaagttagcctatcccaccgttgcttcctacacgtgtcgaccatccggaccatatatagacagctataccacacccgcgtggatgcatgcatgggtcggtcatcccttacttgtgatgtatagacaattcacactaatataatatatatataacttgctttgatttttgatctctgatatatatcgccagcatattatcactgcctccactacttatcctcgctccatctctcagaaacaataattcaggagccatgcatgtagccatatcctccctaggtcgttctgttcattcctgcattaaaaaaatgtcaattgtaatgggtctaaaggtgcaagtaagtttgagattataatatggagagtttatatagttcgaacattagtatcgtcgttttttatttgcaatagattctaatgtaactctttagtaccggttgtaacaaaaaaaccagtactaaatactaagttttcaacaacaactgatactaaaattattttattagttttctacaaacagtatgatatcgacaaagagtacattaataattatgtgcgtatacaaatatacaagttctttccaatgaatatgtgcacatacaaaaatacaagttctttccaatagatgtgggcggatacaaacatacaaatatccaaatatacatatagcttctttattgaatatacaaatcttaggagagtattatatatttttagtacaaggaaagtgcgaaaacacgtctgatttccttcttctctattttttcgtctcagttccttctgccctaacctactatgtactgcaaccctacttcctcttgtgcggaggttttaggtcaagggattccaaaccaacaggtttaatgtgagggacgaagatgctcttacaaccaactagcccttgaagcaggaaatagaatgtattttcttatttcctgccataggactagttcatttttcaagatcttctccatttcactataaccaatgcgaggatggtttctatgaaaaatatacaaaacagaaaaaatagagttcaaaacaaatggtttaatagttatttatggacagtaagcaaaaaataaaaaaacatggatgaatatcattttgcgactgtgtaaagaatactcgaggcacgtgacatttgtgttgtactgatacttaacacactatgtgacatttactcaagactttcattggtactcaacacgtccagtgctcacgaatgagaaaggcaggctgcgcttataggagacacccctagccaataacttgtactaaatccttcacttttagtgttggttatttaaaagaagcggtactaaactaattttggcgggaattccaattgctctggtaggattggaaaatatctttctatatcggttcttataaaaacggtactaaagagttaaaatttagtatcgattctataaaagaaccgatactaaagatattggggatgttctcttcgaattaaagccgactatttggactgctgcaactgtaatccatagagaaaaaaacactatggaagcttggtacagattaaagtcaagcctaattaatgatggttcttaggttgaactagcagtgctagataacttaataatatgtattggttgcagacaacaaccggtactaatatcctctgcacggactatattttaaaacggtgctaaagctagtactaatattggaacaaatataattggacggaaaccccattctccggtagggaataatgatttgtttttcttcttgcaatttaatttgtttagtttatattctcgctagcttgacgacataaatacatgtgtagatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacacagcggatatacctctcgatcaaccgcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccctagctggtgatgagaacgacgaccagcctgcgtttcacccgtttgggacaccacagtaccaccaccccagaaaagggaacgggaacgggaacgggctcacctttgagctggacgccacgctgggcctcggcaccgcgcggcaaaccactgagacagcagaagcaagcgccaccatcgtaagtattgctcccgaattatcttaaataagttcagataattcacatgcatggtttctaattggaatttggtcccaagctggactaccttttttttcttccgttttctcaactctcttatcgatcacctgcataaaggacctttgtatcaagtaccaagagaccttgccatgagttgcactttacgcacatattttttttcttttttttcaggaacgtactactcttcctatatatcaatatatgtaaacaagattaacatgcatgtttctaacctttctcaaagacaaaagacaccctggtgcacgaaatggatggaagaaaccagatcattaatatatgcctcacaaccttcatgaatttaatttgatgtggaaagaataaaaacgacggttccggttgttaacccaatattcaatgatatcctgaacaaaactagctatagatctcaatcatagcatcaggcatcagcgcttccaaagttctcacctgacctttttttttactcaatctccagattatatttccttcctacaaaagagttggagagaacatatatagccatatgagttaaatcgctgatgttttaaatacagaccagtagtcaaaagcattgactatacactaaaactattgttcaaggtcactatttcacacaaaatttcattgcctattatttgatagtttgattgagaggtagcaatattgccaaatttatttttcacgtacctcgacaaaagtcggtagcaatattgccaattttattgctccatcttcatatccagcccgaagtctattattgctgtaatgacaagatacgtatcttttatattgtgatcgatatacttacttaagttatatattgacgataaaagggagaaagcagcttgcctccctttctttttcttcaccactatatatattggattgtttcttcaccacaatatacaagaaaatattaatatctgcagtacatatttggtgtcattaaatatgtcgtttgaaactattttcataataagcatatttgaagatacagatattgcaaatattttttacgaatctaatcaaatatgagaaattttgactgacatgtatgaccatactatcaattattttaggacagaggcaggaagtgcgcatttgtatggtcgaaatcgatcaattgtaaccatatgcatgtacgttggtacgcccactgatgtatctacctggttaattaattagatgacctagcttgtcgtctgattgttatatatgattaaagaaccaaaaagtctactcagctcaaaacccaaatatatatgtctcaaacaactcccatgcacatgtccagctgtgtctaaatctatcccgaaggattgtccatgccaaagtttgatgaaataggtaataagttgtctcattttatgtggttaatttgcagatttgctgcttactctttggtatacttggattttatagggaactaatatatacatatgattatgattaatgcactttatttcgtgccacatgtagatgaataactcaatcacatggcttaagatctaatattctaccccccaaaaacaaatggtgctaccaaggcaatatctgatgttcatcaggcatgcatgtagctaggcccattcagcatatcaagcaaagtacagattcttatccaaacaatatatatgaccaaagtactaattaattagttgcctgcagttagctgtccaaaatttgctttgatcatcatgcaataatatacacatgcagaaactaaaatgcaataacatatataaatacatgcatgcatgaacatgcatactaattgctattaaataattgcagatgtcattctgtgggagcacattcactgacgctgcaagcaaggagccagcactgatcgacgacggcaatgagctgcaaatgccggtagatcagtcgtcgtcggagagggaggttaagttgatgaggtacaaggagaagagaatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacggctgaatcttatgtatatatgtatttatagttagtaaatgtattactccctctattcttttttatttgtcaccgattagtttaaaaattaactagcggacgacaaatattcgagaacagagatagtatatgttatgtctcaattgccaactgcaatagcaaaacttttggggatact

SEQ ID NO: 35 - DNA, 726 bp, Zea mays spp parviglumis
atgtcgtcggggccagcagcatgcggtgtgtgcggcgcggccgcctgctgccggcacctcttgcacaccggtggcagcaacgacgacgacctcatcagccgggccttcttctctgtcttccctgtcgtcggtcatcaccgtcgtcatgagtccaccagcagcctcgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccctagctggtgatgagaacgacgaccagcctgcgtttcaaccgtttgggacaccacagtaccaccaccccagaaaagggaacgggaacgggaacgggctcacctttgagctggacgccacgctgggcctcggcaccgcgcggcaaaccactgagacagcagaagcaagcgccaccatcatgtcattctgtgggagcacattcactgacgctgcaagcaaggagccagcactgatcgacgacggcaatgagctgcaaatgccggtagatcagtcgtcgtcggagagggaggttaagttgatgaggtacaaggagaagagaatgaggaggtgctttgagaagcagataagatatgcatccaggaaagcctatgcgcaggtgagacccagggtgaaaggccgctttgccaaggtaaccgaa

SEQ ID NO: 36 - protein, 242 aa, Zea mays spp parviglumis
MSSGPAACGVCGAAACCPHLLHTGDGNDDDLISRAFFSVFPVVGHHRRHESTSSPAMQQPSGCLHEFQFFGHQDDHHHQETIAWLLDHPPPPAPELGGDDGPSLAGDENDDQPAFHPFGTPQYHHPRKGNGNGNGLTFELDATLGLGTARQTTETAEASATIMSFCGSTFTDAASKEPALIDDGNELQMPVDQSSSEREVKLMRYKEKRMRRCFEKQIRYASRKAYAQVRPRVKGRFAKVTE

SEQ ID NO: 37 - DNA, 4798 bp, Zea luxurians
gcggattctgtttctgtgtaaacaaccaacactctctttattccaatggacaatgctccatctctcttgtgccccccgcaggacagaattggctaggcggcgattccacgtgccatcacatttccaattctgaagttagcctatcccaccgttgtttcctacacgtgtcgaccatccggccgaccatatatagacagctaccacacccgcgtggatgcatgcatgggtcggtcatcccttgtgatgtatagacaattcacactaatataatatataatattcaggagcatgtacactactcagtttctttaccgagtgtccgacaaaaaacactcggcaaagagccggacactcggtaaaaacccggattccggtagtggtagtcatatccttcctagattgttctgttcattcctgcattaaaaaatgtcaattgtaatattgcaaataagtttgagattataacacggagagtacattaataattatttgcatatacaaatatacaagttctttccaatggatatgtgcacatacaaaaatacaagttctttccaatagatatgagcagatacaaatatccaaatatacatatagcttctttattaaatatataaatcttaggggagtattatatatctttagtacaaggaaagtgcgaaaaaacatctgattcccttcttctctttttttttcgtctcagttccttctgtcctaacctactacgtactacacccctacttcctcttgtgcggaggttttaggtcaagggattccaaaccaacagttttaatgtgagggacgaagatgctcttacatccaactagcccttgaagcaaaaaatagaatgtattttcttatttcctgtcatagggctagttcatttttcaagatcttctccatttcactatgaccaatgtgaggatggtttctatgaaaacatatataaaacagaaaaaatagagttcaaaacaaatggtgtaatagttatatatggacactaaacaaaaaataaaaaacatggatgaatatcattttgcgactgtgtaaaaaatactcaaggcacatgacatttgtgttgtactgatacttaacacactatatgtgacatttactcaaaattttcagtggtactcaacacgtccagtgctcacgaatgagaaaggcaggctgcgcttataacacccctagccaataacttgtactaaatccttcacttttagtgttggttatttaaaagaagcagtgctaaactaattttggcgggaattccaattgctctcgcaggattggaaaatatctttctatatcggttcttataaaaacggtactaaaggcatctctattttttgagtacttgttcttgaggtactaaataaatgcaatatttccaaccagtactaaagagttaaaatttagtatcgattctataaaaaaaccgatactaaaaatattggggatgttctctttgaattaaagccgactatttggactgctgtaactgtaattcataatctgtggaagcttggtacagattaaagtcaaacctaattagtgttggttcttaggttgaactagccatgctagataacttaataataattattggttgcagacaacaatcgatactagtatcctctgcacggactatattttaaaacggtgctgaagtcagtactaatattggaacgaatataattggacggaaaccccattctctggtagggaataatgatttgtttttcttcttgcaatttaatttgtttagtttatattctcgctatgttgacgacataaatacatgtgtagatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaagtaactagccctagcagctagctatcaagctttatatctgctccttcctccatctccctgctggcctgcctgatcagtatatataactctcaattccatcaacaaatctccctcccagctagcagtcgatccatcttgtgcacacacagcggatatacctctcgatcaacagcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtccgtcgtcatgagtccaccagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagccggcgtttcacccgtttgggacaccacagtaccaccaccccacaaaagggaacgggaacgggaacgggctcacctttgagctggacgccaggctgggcctcggcaccgcgcggcaaactgagacagaagcagcaagcgccaccatcgtaagtattgctcccgaattatcttaagtaagttcagataattcacatgcatggtttctaattggaatttggtcccaagctggacaacctttttttttcttccgttttctcaactctcttaaaggacctttgtatcaagtaccaacagatcttgccatgagttgcactttacgcacatattttttttctttttttcaggatcgtactactcttcctatatatgtaaacaagattaacatgcatatatgtttctaacctttctcaaagacaaaagacactctggtgcacgaaatggatggaagaaaccagatcattaatatatgcctcacaaccttcatgaatttaatttgatgtggaaagaataaaaacgatggtaccggttgatgttaacccaatattcaatgatatcctgaacaaaactagctatagatctcaatcatagcatcagcgcttccaaagttctcacctgacttttttttactcaatctccagattatgtttccttcctacaaagagttggagagaacgcatagccatgagttaaatcactgatgttgcaaatacagaccagtagtcaaaagcattgactatacactaaaactattgttcaaggtcactatttcacacaaaatttcattgcctatttgatagttatgagaggtaaagtccatatgcataagaaatacgatttatttttcacgtacctagcagacaaaagtcggtagcaatactgccaattttattgctccatcatcatatccagcccgaagtctattgctgtaatgacaagatacagatcttttatattgtgatatacttacttaagttatatatttgacgataaaagggagaaagcagcttgcctccctttctttttcttcaccactatatatattggattgtttcttcaccactatatacaagaaaatattaatacctgtagtacatatttagtgtcattaaatatgtcttttgaaactattttcataataaacatatttgaagatacagatattgcaaatattttttacgaatctaatcaaatatgagaaattttgactgacatgtatgaccatactatcaattattttaggacagaggcagggagtgcgcatatttttacggtcgaaatcgatcaattgtaaccatatgcatgtacgttggtacgcccactgatctatctacctggttaattaattagatagatgacctagcttgtcgtctgattgttatgattaaagaaccaaaaggtgtactcaaaacccaaatatatatgtgtcaaacaattcccatgcatgcacatgtccagctgtgtctaaatctatcccgaaggactgtccatgccaaagtttgatgaaataggtaataagttgtctcattttatgtggttcatttgcagatttgcttcttactctttggtatacttggattttatagggaactaatatatacaaatgattatgattaatgcaatttatttcgtgccacatgtagatgatgaacaactcaatcacatggcttaagatctaattaatattctatcccccaaacaaatggtgctaccaaggcaatatctgatgttcatcaggcatgcatgtaggcccattcagcatatcaagcaaagtacagattcttatccaaaccatatatatatgaccaaagtactaattaattagttgcctgcagttagctgtccaaaatttgctttgatcatcatgcaatatacatatgcccgcagaaactaaaatgcaataacatatataaattcatgcatgcatgcacatgcatactaattgctattaattaattgcagatgtcattctgtgggagcacattcacggacgctgcaagcaaggagccagcactgatcgacgacggcaatgagctgcaaatgccggtagatcaggcgtcgacggagagggaggttaagttgatgaggtacaaggaaaagaggatgaggaggtgctttgagaagcagataagatatgcatccaggaaagcctatgcgcaggtgagaccccgggtgaaaggccgctttgccaaggtaaccgaatgacgacgaagcctgctagctaagtactccgccacagcagacgacgacgacgactgaatccatagctagtaaatgtagtatatgttatgtgtcaattgtcgactgcaatagcaaaacttttggggatact

SEQ ID NO: 38 - DNA, 711 bp, Zea luxurians
atgtcgtcggggccagcagcatgcggtgtgtgcggcgcggccgcctgctgcccgcacctcttgcacaccggtgacggcaacgacgacgacctcatcagccgggccttcttctccgtcttccctgtcgtccgtcgtcatgagtccaccagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccccagctggtgatgagaacgacgaccagccggcgtttcacccgtttgggacaccacagtaccaccaccccacaaaagggaacgggaacgggaacgggctcacctttgagctggacgccaggctgggcctcggcaccgcgcggcaaactgagacagaagcagcaagcgccaccatcatgtcattctgtgggagcacattcacggacgctgcaagcaaggagccagcactgatcgacgacggcaatgagctgcaaatgccggtagatcaggcgtcgacggagagggaggttaagttgatgaggtacaaggaaaagaggatgaggaggtgctttgagaagcagataagatatgcatccaggaaagcctatgcgcaggtgagaccccgggtgaaaggccgctttgccaaggtaaccgaa

SEQ ID NO: 39 - protein, 237 aa, Zea luxurians
MSSGPAACGVCGAAACCPHLLHTGDGNDDDLISRAFFSVFPVVRRHESTSPAMQQPSGCLHEFQFFGHQDDHHHQETIAWLLDHPPPPAPELGGDDGPSPAGDENDDQPAFHPFGTPQYHHPTKGNGNGNGLTFELDARLGLGTARQTETEAASATIMSFCGSTFTDAASKEPALIDDGNELQMPVDQASTEREVKLMRYKEKRMRRCFEKQIRYASRKAYAQVRPRVKGRFAKVTE

SEQ ID NO: 40 - DNA, 2980 bp, Zea mays spp Mexicana
tctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatcttgtgcacacacagcggatatacctctctatcgatcaacagcggccatgtcgtcggggccagcagcatgcggtgtgtgcggcgcggccgcctgctgcccgcacctcttgcacaccggtgacggcaacgacgacgacctcatcagccgggccttcttctccgtcttccctgtcgtcggtcatcaccgtcgtcatgagtccaccagcagccccgccatgcagcagccatcggggtgcctgcacgagttccagttctttggccatcaggacgaccaccaccaccaagaaaccatcgcctggctcttggaccacccaccgccacctgcgcccgagcttggcggcgacgacggcccgtccctagctggtgatgagaacgacgaccagcctgcgtttcacccgtttgggacaccacagtaccaccaccccagaaaagggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggacaccctttttttatcttccgttttctcaactctcttatcgatcacctgcataaaggacctttgtatcaagtaccaagagatcttgccatgagttgcactttacgcacattttttttttctttttttttcaggaacgtactactcttcctatatatcaatatatgtaaacaagattaacatgcatgtttctaacctttctcaaagacaaaagacactctggtgcacgaaatggatggaagaaaccagatcattaatatatgcctcacaacctcttcatgaatttaatttgatgtggaaagaataaaaacgacggttccggttgttaacccaatattcaatgatatcctgaacaaaactagctatagatctcaatcatagcatcaggcatcagcgcttccaaagttctcacctgacttttttttactcaatctccagattatatttccttcctacaaagagtcggagagaacatagccatgagttaaatcactgatgttgtaaatacagaccagtagtcaaaagcattgactatacactaaaactattgttcaaggtcactatttcacaaaaaaattcattgcctatttgatagtttgattgagaggtagcaatattgccaaatttatttttcacgtacctagacaaaagtcggtagcaatattgccaattttattgctccatcgtcatatgcatcccgaagtctattattgctgtaatgacaagatacagatcttttatattgtgatatacttacttaagttttatattgaagataaaagggagaaagcagcttgcctccctttctttttcttcaccactatatatattggattgtttcttcaccactatatatacaagaaaatattaatatctgcagtacatatttagtgtcattaaatatgtcttttgaaactattttcataataaacatatttgaagatacatgtattgcaaatattttttacgaatctaatcaaatatgagaaattttgactgacatgtatgaccatactatcaattattttaggacagaggcatggagtgcgcatttgtatggtcgaaatcgatcaattgtaaccatatatgcatgtacgtttggtacgcccactgatgtatctacctggttaattaattagatgacctagcttgtcgtctgattgttatgattaaagaaccaaaaagtctactcagttcaaaacccaaatatatatgtgtcaaacaactcccatgcacatgtccagctgtgtctaaatctatcccgaaggattgtccatgccaaagtttgatgaaatagataataagttgtctcattttatgtggttcatttgcagatttgctgcttactctttcgtatacttggattttatagggaactaatatatacatatgattataattaatgcactttattccgtgccacatgtagatgaataactcaatcacatggcttaagatctaatattctaccccaaaacaaatcgagctaccaaggcgatatctgatgttcatcaggcatgcatgtaggcccattcagcatatcaagcaaagtacagattcttatccaaaccatgcatatatatatgaccaaagtactaattaattagttgcctgcagttattagctgtccaaaatttgctttgatcatcatgcaataatatacacatgcagaaactaaaatgaataacatatataaatccatgcatgcatgcagcatactaattattgctattaattaattgcagatgtcattctctgggagcacattcacggacgctgcaagcaaggagccagcactgatcgacgacggcaatgagctgcaaacgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctagctccaccacagcagacgacgacgcttgaatcttatgtatatatgtatgtatagttagtaaatgtagtactctctctattcttttttatttgtcgccgattagtttaaaaataaactagcggacgacaaatattcgagaacagaggtagtatatgttatgtctcaattgccaactgcaatagcaaaacttttggggatact

SEQ ID NO: 41 - DNA, 723 bp, Zea mays spp Mexicana
atgtcgtcggggccagcagcatgcggtgtgtgcggcgcggccgcctgctgccggcacctcttgcacaccggtggcagcaacgacgacgacctcatcagccgggccttcttctccgtcttccctgtcgtcggtcatcaccgtcgtcatgagtccaccagccccgccatgcagcagccatcggggtgcctgcacgagttccagttctttggccatcaggacgaccaccaccaccaccaagaaaccatcgcctggctcttggaccacccaccgccacctgcgcccgagcttggcggtgacgacggcccgtccctagctggtgatgagaacgacgaccagcctgcgtttcacccgtttgggacaccacagtaccaccaccccagaaaagggaacgggaacgggaacgggctcacctttgagctggacgccacgctgggcctcggcaccgcgcggcaaactgagacagcagaagcaagcgccaccatcatgtcattctctgggagcacattcacggacgctgcaagcaaggagccagcactaatcgacgacggcaatgagctgcaaatgccggtagatcagtcgtcgacggagagggaggttaagttgatgaggtacaaggagaagaggatgaggaggtgctttgagaagcagataagatatgcatccaggaaagcctatgcgcaggtgagacccagggtgaaaggccgctttgccaaggtaaccgaa

SEQ ID NO: 42 - protein, 241 aa, Zea mays spp Mexicana
MSSGPAACGVCGAAACCRHLLHTGGSNDDDLISRAFFSVFPVVGHHRRHESTSPAMQQPSGCLHEFQFFGHQDDHHHHQETIAWLLDHPPPPAPELGGDDGPSLAGDENDDQPAFHPFGTPQYHHPRKGNGNGNGLTFELDATLGLGTARQTETAEASATIMSFSGSTFTDAASKEPALIDDGNELQMPVDQSSTEREVKLMRYKEKRMRRCFEKQIRYASRKAYAQVRPRVKGRFAKVTE

SEQ ID NO: 43 - DNA, 5310 bp, Zea mays spp huehuetenangensis
gcggattctgtttctgtgtaaacaaccaacactctctttattccaatggacaatgctccatctctcttgtgccggccccccgcaggacagaattggctaggcggcgattccacgtgccatcacatttccaattctgaagttagcctatcccaccgttgcttcctacacgtgtcgaccatccggaccatatatagacagctataccacacccgcgtggatgcatgcatgggtcatcccttgtgatgtatagacaattcacactaatataatataatatataacttgctttgttttttgatctctgatcgatatatagcggtagcatattatcactgccaccacttatcctcgctccatctctcagaaacaataattcaggagccatgcatgtagtcatgcatatcctccctaggtcgttctgttcattcctgcattaaaaaatgtccattgtaatgggtctaaaggtgtaaacactaccggaatttgggtctttgtcgagtgtcaaattctttgtcgagtgttttttcagacactcgacaaagaattcctttgcccagtgtcacacaaaaaaccctcggtaaaagaaaacactcggcgaaaaagctctttgccgagtgttttatttttgacactaataagtttgagattataatatggagagtttatatagttcgaacattactatcgttttatatttgcaatagattctaatgtaactctttagtaccggttgtaacaaaaaaaaccagtactaaatactaagttttcaacaacaactgatactaaaattattttattagttttctacaaacaatatgatattaacagagagtacattaataattatgtgcatatacaaatatacaagttctttccaatggatatgtgcacatacaaaaatacaagtactttccaatagatatgagcagatacaaacatacaaacatccaaatatacatatatatagcttctttattgaatatataaatcttaggagagtacatattatatatttttagtacaaggaaagtgtgaaaacacgtctgattcccttctctattttttcgtctcagttccttcttccctaacctactactgcaaccctacttcctcttgtgcggaggttaggtcaagggattccaaaccaactacgggtttaatgtgagggacgaagatgctcttacaaccaactagcccttgaagtaggaaatagaatgtattttcttatttcctgccataggactagttcatttttcaagatcttctccatttcactatgaccaatgcgaggatggtttctatgaaaaaaatatacaaaacagaaaaaaatagagttcaaaacaaatggtgtaatagttatatatggacagtaaacaaaaaataaaaacatggatgaatatcattttgcgaatgtgtaaaaaatactcgaggcacgtgacatttgtgttgtattgataattaacacactatgtgacatttactcaaaactttcattggtactcaacacgtccggtgctcacgaatgagaaaggcaggctgcgcttataggagacacccctagccaataacttgtactaaatccttcacttttagtgttggttatttaaaagaagcggtgctaaactaattttggcgggaattccaattgctctggcaggattggaaaatatctttctataccggttcttataaaaaaatggtactaaaggcatctctattttttgagttcttgttcttgaggtactaaataaacgcaatatttccaactagtactaaagagttaaaatttagtatcgattctatagaagaaccgatactaaagatattggagatgttctcttcgaattaaagccggctatttggactgctgtaactatatccatagagagaaaaaacactgtggaagcttggtacagttaaagtcaagcctaattagtgctggttcttaggttgaactagcagtgctagataacttaataataagtattggttgtagacaacaaccggtactaatatcctctgcacggactatattttaaaacggtggtcaagttagtactaatattggaacaaatataattggacggaaaccccattctccggtagggaataatgatttgtttttcttcttgcactttaatttgtttagtttatattctcgctagcttgacgacataaatacatgtgtagatagaacattgcctatatacccagagatggagtctcgtccatgcatatctattcggcagcgggcattggctaccagattctgcgatatggaacatctgcttttctttctgctgcttcgtctctggcctttgcttttccttcactacgacttaaataaaagtaactagccctagcagctagctatcaagctttatttatctgctccttcctccatctccctgctggcctgcctgatcagtatatataactctcaattccatcaacaaatctccctcccaagctagtcgatccatctgcttgtgcacacacagcggatatacctctcgatcaaccgcggccatgtcgtcggggccagcagcatgcggtgtgtgcggcgcggccgcctgctgccggcacctcttgcacaccggtggcagcaacgacgacgacctcatcagccgggccttcttctctgtcttccctgtcgtcggtcatcaccgtcgtcatgagtccaccagcagccccgccatgcagcagccatcggggtgcctgcacgagttccagttctttggccatcacgacgaccaccaccaccaagaaaccatcgcctggctcttggaccacccaccgccacctgcgcccgagcttggcggcgacgacggcccgtccctagctggtgatgagaacgacgaccagcctgcgtttcacccgtttgggacaccacagtaccaccaccccagaaaagggaacgggctcacctttgagctggacgccacgctgggcctcggcaccgcgcggcaaaccactgagacagcagaagcaagcgccaccatcgtaagtattgctcccgaattatcttaagtaagttcagataattcacatgcatggtttctaattggaatttggtcccaagctggactaccttttttttcttccgttttctcaactctcttatcgatcacctgcataaaggacctttgtatcaagtaccaagagaccttgccatgagttgcactttacgcacatatttttttcttttttttcaggaacgtactactcttcctatatatcaatatatgtaaacaagattaacatgcatgtttctaacctttctcaaagacaaaagacaccctggtgcacgaaatggatggaagaaaccagatcattaatatatgcctcacaaccttcatgaatttaatttgatgtggaaagaataaaaacgatggttccggttgttaacccaatattcaatgatatcctgaacaaaactagctatagatctcaatcatagcatcaggcatcagcgcttccaaagttctcacctgacttttttttttactctatctccagattatatttccttcctacaaaagagttggagagagaacatagccatgagttaaatcactgatgttgtaaatacagaccagtcaaaagcattgactatacactaaaactattgttcaagttcactatttcacacaaaatttcattgcctactatttgatagtttgattgagaggtagcaatattgccaaatttatttttcacgtacctagacaaaagtcggtagcaatattgccaattttattgccccatcgtcatattcagcccgaagtctattattgctgtaatgacaagatacagatcttttatattgtgatatacttacttaagttatatatatattgacgataaaagggagaaagcagcttgcctccctttctttttcttcaccactatatatattggattgtttcttcaccactatatatatgaaaatattaatatttgtagtacatattagtgtcattaaatatgtcttttgaaactattttcataataaacatatttgaagatacagatattgcaaatattttttacgaatctaatcaaatatgagaaattttgactgacatgtatgaccatactatcaattattttaggacagaggcagggagtgcgcatttgtatggtcgaaatcgatcaattgtaaccatatgcatgtacgttggtacgcccactgatgtatctacctggttaattaattagatatatgacctagcttgtcgtctgattgttatgattaaagaaccaaaaagtctactcagctcaaaacccaaatatatatgtgtcaaacaactcccatgcacatgtccagctgtgtctaaatctatcccgaaggattgtccatgccaaagtttgatgaaataggtaataagttgtctcattttatgtggttcgtcatttgcagatttgctgcttaatctttggtatacttggattttatagggaactaatatatacatatgattataattaatgcactttatttcgtgccacatgtagatgatgaacaactcaatcacatggcttaagatctaattaatattctaccaacccaaacaaatggtgctaccaaggcaatatctgatgttcatcaggcatgcatgtagctaggcccattcagcatatcaagcaaagtacagattcttatccaacaaacaatatatatgaccaaagtactaattaattagttgcctgcagttagctgtccaaaatttgctttgatcatcatgcaataatatacacatgcagaaactaaaatgcaataacatatataaatccatgcatgcatgcacatgcatactaattattgctattaattaattgcagatgtcattctgtgggagcacattcactgacgctgcaagcaaggagccagcactgatcgacgacggcaatgagctgcaaatgccggtagatcagtcgtcgacggagaggggggttaagttgatgaggtacaaggagaagaggatgaggaggtgctttgagaagcagataagatatgcatccaggaaagcctatgcgcaggtgagacccagggtgaaaggccgctttgccaaggtaaccgaatgacgaagcctgctagctaactccgccacagcagacgacgacgactgaatccatagtatgtatttatatgtatgtatagttagtaaatgcagtactcccctgttcttttttatttgtcaccgattagtttaaaaataaactagcggacgacaaatattcgaaaacagaggtagtatatgttatgtctcaattgccaactgcaatagcaaaacttttggggatact

SEQ ID NO: 44 - DNA, 714 bp, Zea mays spp huehuetenangensis
atgtcgtcggggccagcagcatgcggtgtgtgcggcgcggccgcctgctgccggcacctcttgcacaccggtggcagcaacgacgacgacctcatcagccgggccttcttctctgtcttccctgtcgtcggtcatcaccgtcgtcatgagtccaccagcagccccgccatgcagcagccatcggggtgcctgcacgagttccagttctttggccatcacgacgaccaccaccaccaagaaaccatcgcctggctcttggaccacccaccgccacctgcgcccgagcttggcggcgacgacggcccgtccctagctggtgatgagaacgacgaccagcctgcgtttcacccgtttgggacaccacagtaccaccaccccagaaaagggaacgggctcacctttgagctggacgccacgctgggcctcggcaccgcgcggcaaaccactgagacagcagaagcaagcgccaccatcatgtcattctgtgggagcacattcactgacgctgcaagcaaggagccagcactgatcgacgacggcaatgagctgcaaatgccggtagatcagtcgtcgacggagaggggggttaagttgatgaggtacaaggagaagaggatgaggaggtgctttgagaagcagataagatatgcatccaggaaagcctatgcgcaggtgagacccagggtgaaaggccgctttgccaaggtaaccgaa

SEQ ID NO: 45 - protein, 238 aa, Zea mays spp huehuetenangensis
MSSGPAACGVCGAAACCRHLLHTGGSNDDDLISRAFFSVFPVVGHHRRHESTSSPAMQQPSGCLHEFQFFGHHDDHHHQETIAWLLDHPPPPAPELGGDDGPSLAGDENDDQPAFHPFGTPQYHHPRKGNGLTFELDATLGLGTARQTTETAEASATIMSFCGSTFTDAASKEPALIDDGNELQMPVDQSSTERGVKLMRYKEKRMRRCFEKQIRYASRKAYAQVRPRVKGRFAKVTE

SEQ ID NO: 46 - DNA, 864 bp, Oryza sativa
atggggatggccaatgaggagtcgccaaattatcaggtgaaaaaaggcggccggattcctccacctcgatcgagtttgatttatccgttcatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatag

SEQ ID NO: 47 - protein, 257 aa, Oryza sativa
MSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGGRPDLDAGLGLGGGSGRHAEAAASATIMSYCGSTFTDAASSMPKEMVAAMADVGESLNPNTVVGAMVEREAKLMRYKEKRKKRCYEKQIRYASRKAYAEMRPRVRGRFAKEADQEAVAPPSTYVDPSRLELGQWFR

SEQ ID NO: 48 - DNA, 894 bp, Oryza sativa
atgtcggcggcgtcgggcgccgcgtgcggggtgtgtgggggaggggtgggggagtgcgggtgcctgctgcatcagcggcgtgggggaggcggtggtggtggaggtggaggggtgaggtgcgggatcgcggcggacctgaaccgggggtttccggcgatctttcagggggtgggggtggaggagacggcggtggaaggggatggaggagcccagccggcggccgggctgcaggagttccagttcttcggccacgacgaccacgacagcgtcgcgtggctcttcaacgacccggcgccgcccggcgggacggaccaccagcttcaccgccaaaccgcgcccatggcggtcggcaacggcgcggcggcggcgcagcagcggcaggcgttcgacgcgtacgcgcagtaccagccggggcacgggctcacgttcgacgtgccgctcacccgaggcgaggccgccgccgcggtgctcgaggccagcctcggcctcggcggcgccggcgccggcggcaggaacccggcgacgtcgagcagcacaatcatgtccttctgtgggagcacgttcactgacgccgtgagctccatcccgaaagatcacgcggcggcggcggcggtcgttgccaacggcggcctgagcggcggcggcggcgacccggcgatggaccgggaggcgaaggtgatgcggtacaaggagaagaggaagcggaggcgatacgagaagcagatccggtacgcctcgcgcaaggcctacgccgagatgcggccgcgcgtgaagggccgcttcgccaaggtgcccgacggcgagctggacggcgcgacaccgccgccgccgtcctccgccgccggcggcggctacgagcccggccggctcgacctcggatggttccgttcgtag

SEQ ID NO: 49 - protein, 297 aa, Oryza sativa
MSAASGAACGVCGGGVGECGCLLHQRRGGGGGGGGGGVRCGIAADLNRGFPAIFQGVGVEETAVEGDGGAQPAAGLQEFQFFGHDDHDSVAWLFNDPAPPGGTDHQLHRQTAPMAVGNGAAAAQQRQAFDAYAQYQPGHGLTFDVPLTRGEAAAAVLEASLGLGGAGAGGRNPATSSSTIMSFCGSTFTDAVSSIPKDHAAAAAVVANGGLSGGGGDPAMDREAKVMRYKEKRKRRRYEKQIRYASRKAYAEMRPRVKGRFAKVPDGELDGATPPPPSSAAGGGYEPGRLDLGWFRS